(19) United States
(12) Patent Application Publication
(10) Pub. No.: US 2025/0368672 A1
(43) Pub. Date: Dec. 4, 2025

(54) IRON COMPLEXES AND USES THEREOF

(71) Applicant: Regents of the University of Minnesota, Minneapolis, MN (US)

(72) Inventors: Valérie Christine Pierre, Minneapolis, MN (US); Sheng-Yin Huang, Minneapolis, MN (US)

(73) Assignee: Regents of the University of Minnesota, Minneapolis, MN (US)

(21) Appl. No.: 18/875,498

(22) PCT Filed: Jun. 16, 2023
(86) PCT No.: PCT/US2023/025613

§ 371 Date: Dec. 16, 2024

Related U.S. Application Data
(60) Provisional application No. 63/353,332, filed on Jun. 17, 2022.

(51) Int. Cl. C07F 15/02; C02F 1/68; C02F 101/10

(52) U.S. Cl. CPC C07F 15/025; C02F 1/683; C02F 2101/105

(57) ABSTRACT

Disclosed herein are iron complexes and salts thereof comprising a compound of formula (I) as well as materials and devices comprising one or more iron complexes or salts thereof of formula I. Also disclosed herein are methods of using the iron complexes of formula I or salts thereof, such as methods for detecting ions such as phosphate, methods for removing ions such as phosphate from aqueous solutions or mixtures, and methods for treating hyperphosphatemia.

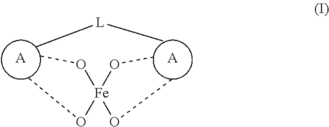

### RELATED APPLICATIONS

This application claims priority to U.S. Provisional Application No. 63/353,332 that was filed on Jun. 17, 2022. The entire content of the application referenced above is hereby incorporated by reference herein.

### GOVERNMENT FUNDING

This invention was made with government support under DK124333 awarded by the National Institutes of Health. The government has certain rights in the invention.

### BACKGROUND OF THE INVENTION

Phosphate is a crucial component of fertilizers needed to maintain the world's food supply. Unfortunately, most of the phosphate used as fertilizer leaches out into surface water, causing widespread eutrophication and hazardous algal blooms. Over 65% of US estuaries and coastal waters now have moderate to severe eutrophication, with significant consequences to the ecology and industry relying on those systems^{1,2}. Addressing this issue requires in part facile detection of phosphate in the μM range^{3,4}.

The current protocol of the US Environmental Protection Agency (EPA) for measuring phosphate levels, the molybdenum blue method, relies on the formation of a phosphomolybdate Keggin ion followed by its reduction to yield a blue mixed-valence complex.⁵ The slow kinetics of these reactions renders this multi-step protocol laborious. Moreover, the strong acidic conditions necessary for the formation of the Keggin ion does not enable distinction between orthophosphate and other polyphosphates such as pyrophosphate that can also be present in large concentration in surface water but have different impact on algae growth.⁶ As such, although much attention has recently been devoted to developing molecular receptors and fluorescent probes for phosphate, effective probes that can readily distinguish between phosphate and pyrophosphate are still needed.⁷⁻¹²

Metal complexes are particularly well-suited for probing phosphates by luminescence. Recognition of the anion can be accomplished either allosterically or via direct coordination. As in the case of the heteroditopic ruthenium(II) bipyridyl complexes, allosteric recognition of phosphate is primarily accomplished by directed hydrogen-bonding interactions. Such probes, however, do not work well with aqueous samples and are rarely selective for phosphate, including over pyrophosphate.¹³⁻¹⁵ Direct coordination of phosphate is better suited for such applications since the metal ions are able to overcome the high hydration enthalpy of phosphate.^{7-11,16,17} The requirements for lability and hardness have limited current studies to copper, zinc, and lanthanide complexes,^{18-23,7} some of which have marked selectivity and affinity for phosphate. Unfortunately, although many of those probes are selective for phosphate over competing anions such as bicarbonate and chloride, selectivity for orthophosphate over polyphosphates such as pyrophosphate has not yet been established.

The presence of iron in the active site of many phosphodiesterases and phosphatases suggest that iron could also be used in the design of receptors for phosphate.²⁴⁻²⁶ Yet, despite being the most abundant transition metal, iron is rarely explored in the design of molecular receptors, as evidenced by the paucity of iron complexes for anion recognition.²⁷⁻³⁰ It is believed that no iron-based molecular receptors for any oxyanion that function at neutral pH and that is selective over interfering anions has been reported.⁷ Despite its hardness appropriate for hard anions, coordinatively unsaturated iron(III) complexes present several challenges for such applications that are not yet fully mastered. In particular, iron(III) complexes with open coordination sites have a propensity to form μ-oxo dimers,^{31,32} which prevents or diminishes further coordination of the targeted anion.³³ The development of Fe^{III}-based receptors for anions thus necessitates a re-engineering of the metal center to prevent such dimerization. In heme-based system, formation of μ-oxo dimers can be prevented by increasing the steric hindrance around the iron center with picket fences^{34,35} or via supramolecular assemblies with cyclodextrins.³⁶ It was thought that in non-heme iron-based systems, coordination at the open site by a weaker anion could be sufficient to prevent dimerization. Given the propensity of Fe^{III} to quench the fluorescence or organic dyes,³⁷ such metal-based receptors would also function as a fluorescent probe if this weak anion also fluoresces.

Other parameters should be taken into consideration in the design of the receptor. First, the affinity of receptors for anions are significantly influenced by the overall charge of the metal complex at the pH of interest.²¹ Highly negatively charged complexes should be avoided. The Fe^{III} complex must also be sufficiently thermodynamically stable to prevent demetallation. The bioinorganic chemistry of siderophores, natural products that are strong iron chelators,³⁸ suggest that both of these requirements can be met with tetra- or pentadentate ligands comprising all oxygen donor such as 1,2-hydroxypyridinone (HOPO). In corresponding molecular receptors Fe^{III}-HOPO-fluo (1) and Fe^{III}-HOPO-PhO-fluo (2) (FIG. 1), the remaining 1 or 2 open coordination sites are protected by fluorescein, a weaker ligand for Fe^{III} than phosphate. It was hypothesized that fluorescein would coordinate sufficiently strongly to iron(III) to prevent formation of μ-oxo dimers, but not too strongly as to enable displacement by phosphate. Accordingly, there is an ongoing need for new metal complexes (e.g., iron complexes) for detecting ions (e.g., anions such as phosphate), for removing ions (e.g., anions such as phosphate)from aqueous solutions or mixtures (e.g., wastewater), and methods for treating hyperphosphatemia.

### SUMMARY OF THE INVENTION

Iron complexes disclosed herein are useful for detecting and sequestering ions (e.g., anions such as phosphate) and may be useful for treating hyperphosphatemia.

Accordingly, one embodiment provides an iron complex composition comprising Fe^{II} or Fe^{III} complexed with two pyridinone ligands, wherein the pyridinone ligands are covalently attached to each other by a linker and wherein each pyridinone is substituted with one hydroxy or —O⁻ and wherein the pyridinone is optionally substituted with one or more (C₁-C₆)alkyl.

One embodiment provides an iron complex comprising a compound of formula I

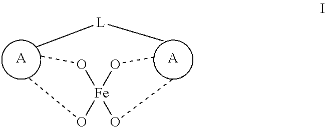

or a salt thereof,
wherein Fe is Fe^{II} or Fe^{III};
each

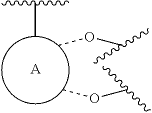

moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.
X^{a} is phenyl substituted with hydroxy or —O⁻; and
W^{a} is a linker^{a} group; and
W^{b} is a linker^{b} group.

One embodiment provides a mixture comprising two or more iron complexes, comprising independently two more compounds of formula I

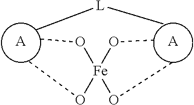

or salts thereof,
wherein Fe is Fe^{II} or Fe^{III} or a combination thereof;
each

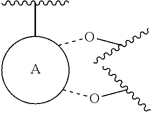

moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.
X^{a} is phenyl substituted with hydroxy or —O—; and
W^{a} is a linker^{a} group; and
W^{b} is a linker^{b} group.

One embodiment provides an iron complex comprising a compound of formula I

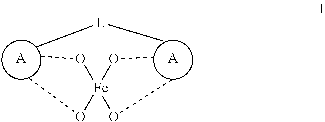

wherein Fe is Fe^{II} or Fe^{III};
each

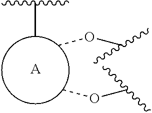

moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y;
X^{a} is phenyl substituted with hydroxy or —O⁻; and
W^{a} is a linker^{a} group;
W^{b} is a linker^{b} group; and
Y is a polymer, hydrogel, membrane, nanoparticle, or material.

One embodiment provides an iron complex consisting essentially of a compound of formula I or a salt thereof as described herein.

One embodiment provides an iron complex of formula I or a salt thereof as described herein.

One embodiment provides a material or device comprising one or more (e.g., 1, 2, 3, 4, 5, 6, 7, 8, 9, 10, or more) iron complexes or salts thereof as described herein.

One embodiment provides a material or device comprising a plurality of iron complexes or salts thereof as described herein.

One embodiment provides a material or device comprising one or more (e.g., 1, 2, 3, 4, 5, 6, 7, 8, 9, 10, or more) iron complexes or salts thereof as described herein, covalently attached to the material or device (e.g., covalently attached through the linker^{a} or linker^{b}).

One embodiment provides a material or device comprising a plurality of iron complexes or salts thereof as described herein, covalently attached to the material or device (e.g., covalently attached through the linker^{a} or linker^{b}).

One embodiment provides a method to detect inorganic phosphate comprising contacting the phosphate with an iron complex as described herein.

One embodiment provides a method to remove inorganic phosphate from an aqueous mixture or solution comprising contacting the aqueous mixture or solution with an iron complex as described herein.

One embodiment provides a method to treat hyperphosphatemia in a mammal (e.g., a human such as a human patient) in need thereof comprising contacting the blood of the mammal in need thereof, with an iron complex as described herein. In one embodiment the mammal has chronic kidney disease.

One embodiment provides processes and intermediates disclosed herein that are useful for preparing an iron complex or a salt thereof comprising a compound of formula I or a salt thereof.

### BRIEF DESCRIPTION OF THE FIGURES

FIG. 1 shows the chemical structures of iron(III)-based luminescent probes for phosphate and detection mechanism. Sol denotes solvent molecules.

FIGS. 2A and 2B show the spectroscopic analyses of Fe^{III}-HOPO-fluo+Pi. FIG. 2A shows the ATR-IR spectra of Fe^{III}-HOPO-fluo and Fe^{III}-HOPO-Pi. FIG. 2B shows the ³¹P NMR of Bu₄N·H₂PO₄* titrated with Fe^{III}-HOPO-fluo (DMSO-d₆, 162 MHz). Experimental conditions: Samples for ATR-IRanalysis was prepared by isolating, rinsing, and drying the precipitate formed from Fe^{III}-HOPO-fluo+1 eq. Pi. [Bu₄N·H₂PO₄]=0.11 M in DMSO-d₆. External reference: 85% H₃PO₄ diluted to 4% with DMSO. *Bu₄N·H₂PO₄ was used due to the low solubility of inorganic phosphate in DMSO, and low solubility of Fe^{III}-HOPO-Pi in CD₃OD.

FIGS. 3A and 3B show the fluorescence titration of Fe^{III}-HOPO-fluo and Fe^{III}-HOPO-PhO-fluo with phosphate (Pi): FIG. 3A shows fluorescence spectra of Fe^{III}-HOPO-fluo with phosphate; FIG. 3B shows increase in emission intensity. Experimental conditions: [Fe^{III}-HOPO-PhO-fluo] and [Fe^{III}-HOPO-fluo]=10 μM in wet ethanol. pH=7. λ_{excitation}=456 nm, excitation and emission slit widths=5 nm, voltage: 600 V. T=25° C. F=integrated fluorescence intensity from 500 nm to 650 nm in the presence of anions, Fₒ=integrated fluorescence intensity in the absence of anions. Fluorescence spectra were obtained 5 min after mixing to ensure that thermodynamic equilibrium was reached. The pH of all solutions was adjusted to 7 carefully by addition of either HCl or NaOH, as necessary.

FIGS. 4A and 4B show fluorescence response. FIG. 4A shows the fluorescence response of Fe^{III}-HOPO-fluo and FIG. 4A shows the fluorescence response of Fe^{III}-HOPO-PhO-fluo to competing anions. White bars represent the relative fluorescence intensity after addition of 1 equivalent of the appropriate anions (NaF, NaCl, NaBr, NaI, Na₂SO₄, NaNO₃, NaHCO₃, NaOAc, Na₄P₂O₇, and Na₂HAsO_{4·}7H₂O). Gray bars represent the relative fluorescence intensity after subsequent addition of 1 equivalent of phosphate (Pi). PPi denotes pyrophosphate: Experimental conditions: [Fe^{III}-HOPO-fluo]=10 μM in wet ethanol, pH 7, λ_{excitation}=456 nm, excitation and emission slit widths=5 nm, F=integrated fluorescence intensity from 500 nm to 650 nm in the presence of anions, Fₒ=integrated fluorescence intensity in the absence of anions. T=25° C. The pH of all solutions was adjusted to 7 carefully using 0.01 N HCl and 0.01N NaOH Fluorescence spectra were obtained 5 min after mixing to ensure that thermodynamic equilibrium was reached. Control denotes the same volume of water was used in replacement of anions.

FIG. 5 shows the HPLC chromatogram of Fe^{III}-HOPO-fluo (1).

FIG. 6 shows the ¹H NMR spectrum of Fe^{III} eIII-HOPO-fluo (1), (CD₃OD, 400 MHz).

FIG. 7 shows the experimental (black) and calculated (red) ESI-MS spectrum of Fe^{III}-HOPO-fluo (1).

FIG. 8 shows the HPLC chromatogram of Fe^{III}-HOPO-PhO-fluo (2).

FIG. 9 shows the ¹H NMR spectrum of Fe^{III}-HOPO-Ph-fluo (2), (CD₃OD, 400 MHz.

FIG. 10 shows the experimental (black) and calculated (red) ESI-MS spectrum of Fe^{III}-HOPO-PhO-fluo (2). The peak at 906.8265 m/z came is from PPG calibrant for obtaining the high-resolution MS spectrum.

FIG. 11 shows the ATR-IR spectra of Fe^{III}-HOPO-PhO-fluo complex in the presence and absence of 1 eq. phosphate. Sample preparations: to the ethanolic Fe^{III} complex solution was added equimolar phosphate. The pH was adjusted to pH=7 using 0.01 N HCl and 0.01N NaOH. The precipitation was centrifuged, rinsed with ethanol, and dried in a vacuum oven.

FIG. 12 shows the ³¹P NMR spectrum of Fe^{III}-HOPO-Pi (DMSO-d₆, 162 MHz). The peak at 0 ppm is the external reference using: 85% H₃PO₄ diluted to 4% with DMSO.

FIG. 13 shows the ³¹P NMR of Bu₄N·H₂PO₄* titrated with Fe^{III}—HOPO-Ph-fluo (DMSO-d₆, 162 MHz). Experimental conditions: [Bu₄N·H₂PO₄]=0.11 M in DMSO-d₆. External reference: 85% H₃PO₄ diluted to 4% with DMSO. *Bu₄N·H₂PO₄ was used due to the low solubility of inorganic phosphate in DMSO, and low solubility of Fe^{III}—HOPO-Pi in MeOH.

FIG. 14 shows the ¹H NMR of Bu₄N·H₂PO₄* titrated with 1 eq. of Fe^{III}—HOPO-fluo+Pi (DMSO-d₆, 162 MHz). Experimental conditions: [Bu₄N·H₂PO₄]=0.11 M in DMSO-d₆. External reference: 85% H₃PO₄ diluted to 4% with DMSO. *Bu₄N·H₂PO₄ was used due to the low solubility of inorganic phosphate in DMSO, and low solubility of Fe^{III}—HOPO-Pi in MeOH..

FIG. 15 shows the Job's plot analysis of Fe^{III}—HOPO-fluo with phosphate. F: integrated luminescence from 500 nm to 650 nm. Conditions: total concentration of Fe^{III}—HOPO-fluo and phosphate was kept at 10 μM. The pH of all solutions was adjusted to 7 using 0.01 N HCl and 0.01 N NaOH. λₑₓ: 456 nm, excitation and emission slit widths: 5 nm, T=25° C. The binding ratios of Fe^{III}—HOPO-fluo+Pi and Fe^{III}—HOPO-PhO-fluo+Pi were estimated by Job's plot studies. Each data point represents the integrated fluorescence emission change with respect to that of same volume of water added in replacement of Pi.

FIG. 16 shows the Job's plot of Fe^{III}—HOPO-PhO-fluo with phosphate. F: integrated luminescence from 500 nm to 650 nm. Conditions: total concentration of Fe^{III}—HOPO-PhO-fluo and phosphate was kept at 10 μM. The pH of all solutions was adjusted to 7 using 0.01 N HCl and 0.01 N NaOH. λₑₓ: 456 nm, excitation and emission slit widths: 5 nm, T=25° C. The binding ratios of Fe^{III}—HOPO-fluo+Pi and Fe^{III}—HOPO-PhO-fluo+Pi were estimated by Job's plot studies. Each data point represents the integrated fluorescence emission change with respect to that of same volume of water added in replacement of Pi.

FIG. 17 shows the kinetics of response of Fe^{III}—HOPO-fluo upon addition of phosphate. F: integrated luminescence from 500 nm to 650 nm in the presence of 1 eq. of phosphate. Conditions: [Fe^{III}—HOPO-fluo]=10 μM in wet ethanol. The pH of all solutions was adjusted to 7. λₑₓ: 456 nm, excitation and emission slit widths: 5 nm

FIG. 18 shows the kinetics of response of Fe^{III}—HOPO—OPh-fluo to phosphate. F: integrated luminescence from 500 nm to 650 nm in the presence of 1 eq. of phosphate. Conditions: [Fe^{III}—HOPO—OPh-fluo]=10 μM in wet ethanol. The pH of all solutions was adjusted to 7. λₑₓ: 456 nm, excitation and emission slit widths: 5 nm..

FIG. 19 shows the UV-Visible and fluorescence titration spectra of Fe^{III} complexes with phosphate: a) UV-Visible spectra of Fe^{III}—HOPO-fluo with phosphate; b) fluorescence spectra of Fe^{III}—HOPO-fluo with phosphate; c) UV-Visible spectra of Fe^{III}—HOPO-PhO-fluo with Pi; d) fluorescence spectra of Fe^{III}—HOPO-PhO-fluo with phosphate. Conditions: [Fe^{III}—HOPO-fluo] and [Fe^{III}—HOPO-PhO-fluo]=10 μM in wet ethanol. pH=7. λₑₓ: 456 nm, excitation and emission slit widths: 5 nm

FIG. 20 shows the ¹H NMR spectrum of intermediate 3, (CDCl₃, 400 MHz).

FIG. 21 shows the ¹³C NMR spectrum of intermediate 3, (CDCl₃, 100 MHz).

FIG. 22 shows the experimental (black) and calculated (red) ESI-MS spectrum of intermediate 3.

FIG. 23 shows the ¹H NMR spectrum of intermediate 4 (CDCl₃, 400 MHz).

FIG. 24 shows the ¹³C NMR spectrum of intermediate 4 (CDCl₃, 100 MHz).

FIG. 25 shows the experimental (black) and calculated (red) ESI-MS spectrum of intermediate 4.

FIG. 26 shows the ¹H NMR spectrum of intermediate 5 (CF₃COOD, 400 MHz).

FIG. 27 shows the ¹³C NMR spectrum of intermediate 5 (CF₃COOD, 400 MHz).

FIG. 28 shows the experimental (black) and calculated (red) ESI-MS spectrum of intermediate 5.

FIG. 29 shows the ¹H NMR spectrum of intermediate 6 (CDCl₃, 400 MHz).

FIG. 30 shows the ¹³C NMR spectrum of intermediate 6 (CDCl₃, 100 MHz).

FIG. 31 shows the experimental (black) and calculated (red) ESI-MS spectrum of intermediate 6.

FIG. 32 shows the ¹H NMR spectrum of protected ligand 7 (CDCl₃, 400 MHz).

FIG. 33 shows the ¹H NMR spectrum of protected ligand 7 (CDCl₃, 400 MHz).

FIG. 34 shows the experimental (black) and calculated (red) ESI-MS spectrum of protected ligand 7.

FIG. 35 shows the ¹H NMR spectrum of ligand 8 (CD₃OD, 500 MHz).

FIG. 36 shows the ¹³C NMR spectrum of ligand 8 (CD₃OD, 125 MHz).

FIG. 37 shows the experimental (black) and calculated (red) ESI-MS spectrum of ligand 8.

FIG. 38 shows the data fitting results of FeIII-HOPO-fluo+Pi and FeIII—HOPO—OPh-fluo+Pi.

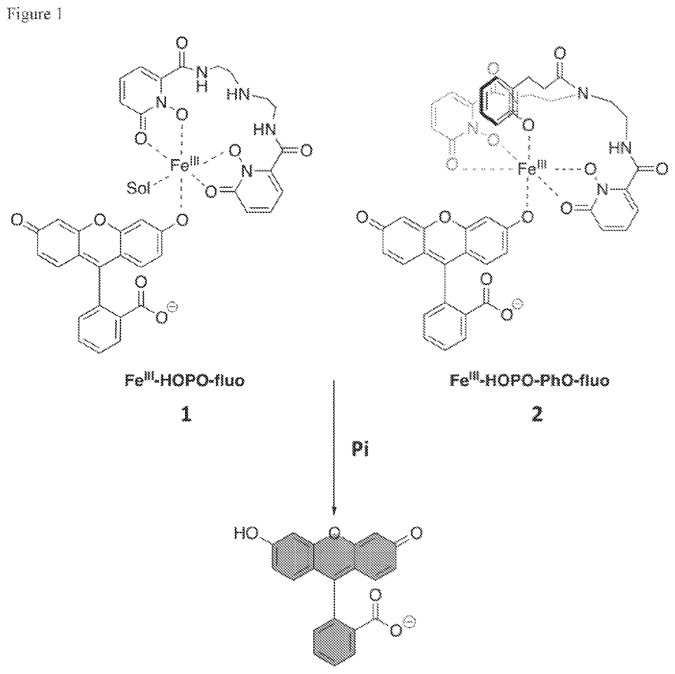

### DETAILED DESCRIPTION

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “alkoxy” refers to —O(alkyl) and the term “haloalkoxy” refers to an alkoxy that is substituted with one or more (e.g., 1, 2, 3, or 4) halo.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

### Moiety

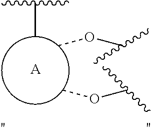

(also referred to herein as “moiety A”)

As used herein “moiety A” is a pyridinone (e.g., oxo(═O) substituted pyridine) this is substituted with one hydroxy or —O—, wherein the pyridinone is optionally substituted with one or more (C₁-C₆)alkyl. In general, the hydroxy is substituted on a carbon atom of the pyridinone and the —O⁻ is substituted on a nitrogen atom of the pyridinone. It is to be understood that the oxygen of the hydroxyl, the oxygen of the pyridinone, and the oxygen of the —O⁻ group coordinate to the iron atom of the iron complex. Thus, the hydrogen of the hydroxyl group is not specifically depicted in the compounds of formula I and may or may not be present in the compounds of formula I.

### Linker (“L”)

As used herein, the linker “L” is a molecular moiety that connects the two “moiety A” groups to one another. The linker can be variable provided it functions to connect two “moiety A” groups to one another, so that the two “moiety A” groups can function as a ligand of the iron metal complexes as described herein. The linker can vary in length and atom composition (e.g., C, H, N, O, S) and for example can be branched or non-branched or saturated or unsaturated or a combination thereof.

In one embodiment L is a linker that comprises 5-50 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-30 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-20 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-15 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 8-15 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 3-15 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-50 non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-30 non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-20 non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-15 non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 8-15 non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 3-15 (or 5-10) non-hydrogen atoms, wherein the non-hydrogen atoms are selected from halo, C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises a branched or unbranched, saturated or unsaturated, hydrocarbon chain, having from 3 to 15 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, oxo (═O), and halo, wherein each R^{a} is independently H or (C₁-C₆)alkyl, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises a branched or unbranched, saturated or unsaturated, hydrocarbon chain, having from 3 to 30 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, oxo (═O), and halo, wherein each R^{a} is independently H or (C₁-C₆)alkyl, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is

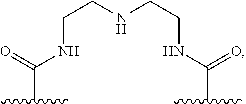

wherein the L is optionally substituted optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y Linker^{a} (“W^{a}”) and linker^{b} (“W^{b}”) (or linker^{a} subgroup and linker^{b} subgroup)

As used herein, the linker^{a} (“W^{a}”) and linker^{b} (“W^{b}”) are molecular moieties that connect the iron complexes described herein (e.g., the compounds of formula I or salts thereof) to another molecular entity such as a material (e.g., polymer (e.g., synthetic or natural polymers), hydrogel, membrane, nanoparticle, or any other suitable material (e.g., a device)). The linker can be variable provided it functions to connect the compound of formula I to another molecular entity, so that both the compound of formula I (e.g., the iron complex) and the other molecular entity can function as described herein. The linker can vary in length and atom composition (e.g., halo, C, H, N, O, S) and for example can be branched or non-branched or saturated or unsaturated or a combination thereof.

In certain embodiments, the material or device includes a membrane has the ligand or the iron complex of the ligand attached thereto. In certain embodiments, the material or device includes a sensor or detector having the ligand or the iron complex of the ligand attached thereto. In certain embodiments, the ligand can be chemically attached to a surface of the material or device (e.g., a surface of the membrane) through covalent and/or ionic bonding using a variety of methods that would be available to one of skill in the art. In certain embodiments, the ligand can include a pendent functional group (e.g., a N, O, P, and/or S-containing group) that can function as a linker to chemically attach the ligand to a surface of the material or device.

As used herein the linker^{a} (“W^{a}”) and linker^{b} (“W^{b}”) may also include one or more reactive groups (e.g., an amine, hydroxy, thiol, ester, or amide; NR₂, OH, SH, CO₂R, CONR₂ wherein each R is independently H or (C₁-C₆)alkyl). These linkers together with the metal complexes can serve as precursors (e.g., intermediates) to which the other molecular entities such as a materials are covalently bonded.

In one embodiment each W^{a} and W^{b} independently comprises 2-50 non-hydrogen atoms, wherein the non-hydrogen atoms are selected from C, N, S, and O.

In one embodiment each W^{a} and W^{b} independently comprises 2-50 non-hydrogen atoms, wherein the non-hydrogen atoms are selected from halo, C, N, S, and O.

In one embodiment each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, hydroxy, and halo, wherein each R^{a} is independently H or (C₁-C₆)alkyl.

In one embodiment each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl.

In one embodiment
W^{a} is

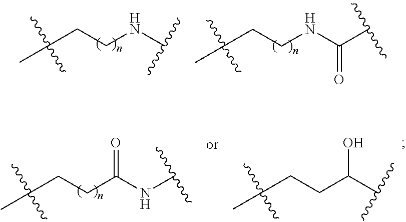

W^{b} is

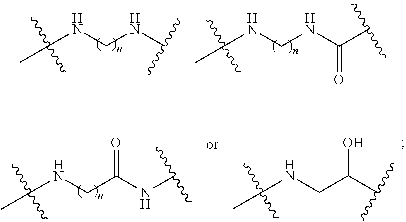

and
n is 1, 2, 3, 4, 5, 6, 7, 8, 9, or 10.

The term polymer includes any polymer suitable for linking to the metal complex described herein (e.g., synthetic or natural polymer). Examples include polyamides (including star polyamides), polyethyleneglycol, polyethylenemine, polysulfone, and polyethersulfone. The term hydrogel includes any hydrogel suitable for linking to the metal complex described herein. Examples include crosslinked poly(N-isopropylacrylamide), crosslinked polyvinyl alcohol, PMA (polymethacrylate), PMMA (polymethylmethacrylate), PEMA (polyethylmethacrylate), and chitosan. The term membrane includes any membrane suitable for linking to the metal complex described herein. The term nanoparticle includes any nanoparticle suitable for linking to the metal complex described herein (e.g., metal-based, silica-based). Examples include gold nanoparticles, iron oxide nanoparticles, and silica nanoparticles. The term material includes any material suitable for linking to the metal complex (e.g., a solid material). Examples include carbon, porous carbon, gold, carbon nanotubes, CuO nanowires, and WO3 nanowires.

### Weak Binding Ligand

As used herein the term weak binding ligand is any ligand that can bind to the iron atom of the iron metal complex but then be subsequently displaced by another ligand or ion (e.g., anion such as phosphate). The weak binding ligand also includes any ligand that can prevent the iron complex from forming iron complex dimers (e.g., iron complexes with two iron atoms).

It is understood that the embodiments provided below or above are for compounds of formula I and all sub-formulas thereof (e.g., formulas Ia, Ib, Ic, Id). It is to be understood that two or more embodiments may be combined.

In one embodiment Fe is Fe^{II}.

In one embodiment Fe is Fe^{III}.

In one embodiment Fe is a mixture of Fe^{II} or Fe^{III}.

In one embodiment each

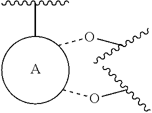

moiety is independently selected from the group consisting of:

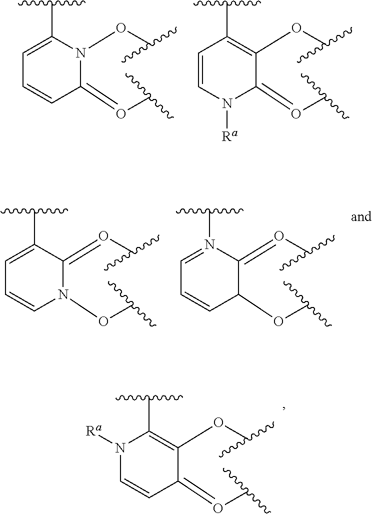

and
wherein each R^{a} is independently (C₁-C₆)alkyl.

In one embodiment each

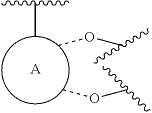

moiety is

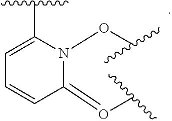

In one embodiment each

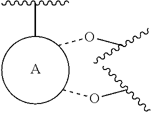

moiety is

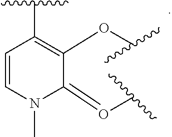

In one embodiment each

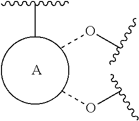

moiety is

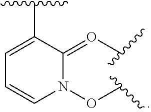

In one embodiment each

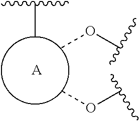

moiety is

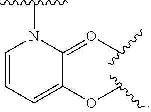

In one embodiment each

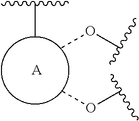

moiety is

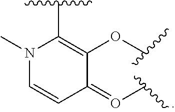

In one embodiment L is a linker that comprises 5-50 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises 5-15 non-hydrogen atoms, wherein the non-hydrogen atoms are selected from C, N, S, and O, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y.

In one embodiment L is a linker that comprises a branched or unbranched, saturated or unsaturated, hydrocarbon chain, having from 3 to 15 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, oxo (═O), and halo, wherein each R^{a} is independently H or (C₁-C₆)alkyl, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y

In one embodiment L is

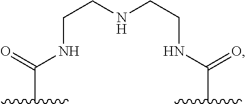

wherein the L is optionally substituted optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y

In one embodiment the compound of formula I is a compound of formula Ia is

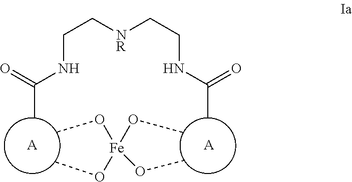

wherein R is H, —W^{a}—Y, or —C(═O)(C₁-C₆)alkyl-X^{a}, and wherein the —C(═O)(C₁-C₆)alkyl-X^{a} is optionally substituted with —W^{b}—Y.

In one embodiment R is H.

In one embodiment R is —W^{a}—Y.

In one embodiment R is —C(═O)(C₁-C₆)alkyl-X^{a}, wherein the —C(═O)(C₁-C₆)alkyl-X^{a} is optionally substituted with —W^{b}—Y.

In one embodiment L is a linker that comprises 5-50 non-hydrogen atoms, wherein the non-hydrogen atoms are selected from C, N, S, and O, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

In one embodiment L is a linker that comprises 5-15 non-hydrogen atoms, wherein the non-hydrogen atoms are selected from C, N, S, and O, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

In one embodiment L is a linker that comprises a branched or unbranched, saturated or unsaturated, hydrocarbon chain, having from 3 to 15 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, oxo (═O), and halo, wherein each R^{a} is independently H or (C₁-C₆)alkyl, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

In one embodiment L is

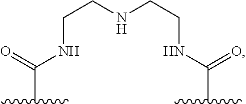

wherein the L is optionally substituted with —W^{a} or —C(═O)(C₁-C₆)alkyl-X^{a}, and wherein the —C(═O)(C₁-C₆)alkyl-X^{a} is optionally substituted with —W^{b}.

One embodiment provides an iron complex or salt thereof comprising a compound of formula Ia

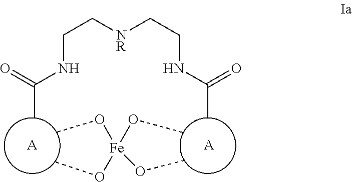

wherein R is H, —W^{a}, or —C(═O)(C₁-C₆)alkyl-X^{a}, and wherein the —C(═O)(C₁-C₆)alkyl-X^{a} is optionally substituted with —W^{b}.

In one embodiment R is —W^{a}.

In one embodiment R is —C(═O)(C₁-C₆)alkyl-X^{a}, wherein the —C(═O)(C₁-C₆)alkyl-X^{a} is optionally substituted with —W^{b}.

One embodiment provides an iron complex or salt thereof comprising a compound of formula Ib

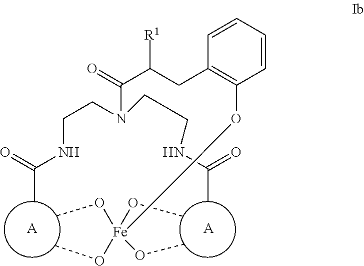

wherein R¹ is H or —W^{b}.

In one embodiment each W^{a} and W^{b} independently comprises 2-50 non-hydrogen atoms, wherein the non-hydrogen atoms are selected from C, N, S, and O.

In one embodiment each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl.

In one embodiment each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, or —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl, wherein the chain is substituted with one or more reactive groups.

In one embodiment each W^{a} and W^{b} independently comprises a branched or unbranched, saturated or unsaturated, hydrocarbon chain, having from 1 to 10 carbon atoms wherein one or more of the carbon atoms is optionally replaced independently by —O—, —S, or —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl, wherein the chain is substituted with one or more reactive groups.

In one embodiment each reactive group is independently an amine, thiol, hydroxy, amide or ester.

In one embodiment
W^{a} is

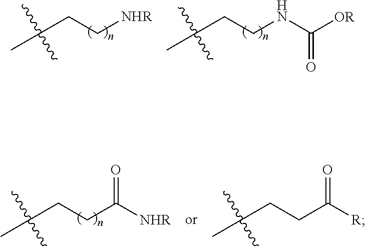

W^{b} is

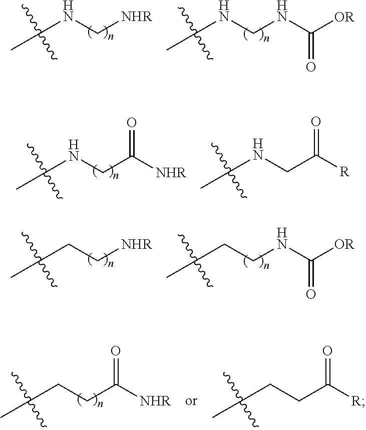

wherein:
R is H or (C₁-C₆)alkyl; and
n is 1, 2, 3, 4, 5, 6, 7, 8, 9, or 10.

In one embodiment the compound of formula I is a compound of formula Ib

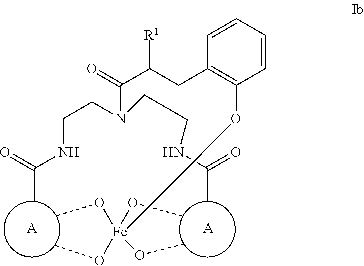

wherein R¹ is H or —W^{b}—Y.

In one embodiment each W^{a} and

W^{b} independently comprises 2-50 non-hydrogen atoms, wherein the non-hydrogen atoms are selected from C, N, S, and O.

In one embodiment each W^{a} and

W^{b} independently comprises a branched or unbranched, saturated or unsaturated, hydrocarbon chain, having from 1 to 20 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, hydroxy, and halo, wherein each R^{a} is independently H or (C₁-C₆)alkyl.

In one embodiment
W^{a} is

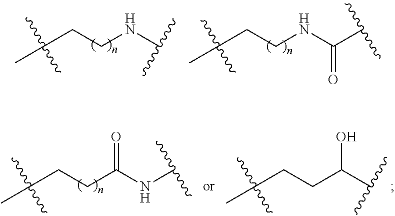

W^{b} is

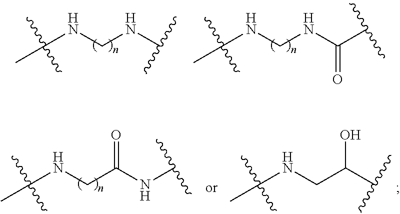

and
n is 1, 2, 3, 4, 5, 6, 7, 8, 9, or 10. 19. In one embodiment

In one embodiment,
W^{a} is

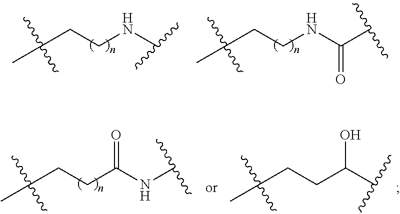

W^{b} is

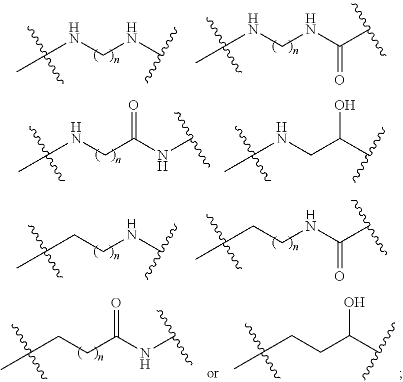

n is 1, 2, 3, 4, 5, 6, 7, 8, 9, or 10.

In one embodiment,
W^{a} is

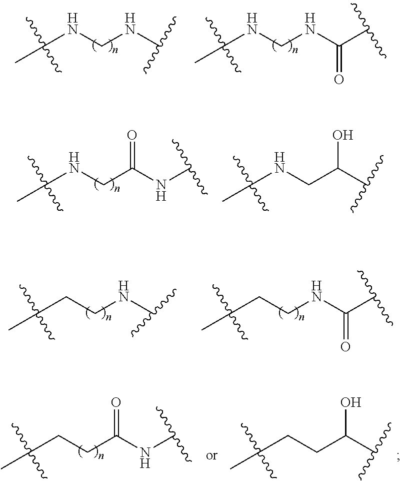

W^{b} is

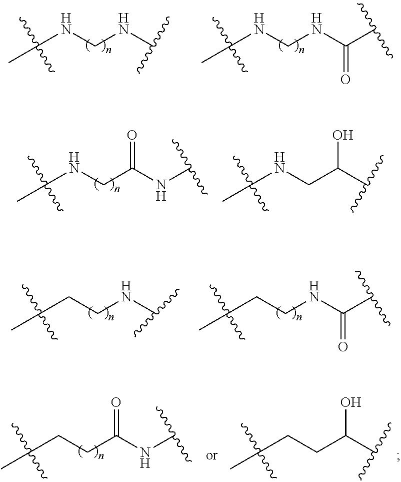

and
n is 1, 2, 3, 4, 5, 6, 7, 8, 9, or 10.

In one embodiment L is

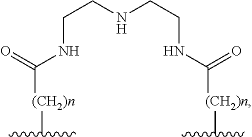

wherein the L is optionally substituted with —W^{a} or —C(═O)(C₁-C₆)alkyl-X^{a}, and wherein the —C(═O)(C₁-C₆)alkyl-X^{a} is optionally substituted with —W^{b} and wherein n is 0 or 1.

In one embodiment the compound of formula I is a compound of formula Ia′

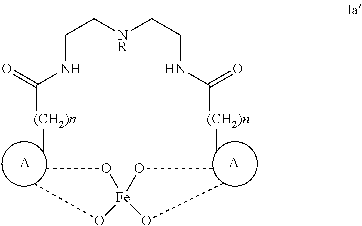

or a salt thereof,
wherein n is 0 or 1, R is H, —W^{a}, or —C(═O)(C₁-C₆)alkyl-X^{a}, and wherein the —C(═O)(C₁-C₆)alkyl-X^{a} is optionally substituted with —W^{b}.

In one embodiment the compound of formula I is a compound of formula Ib′

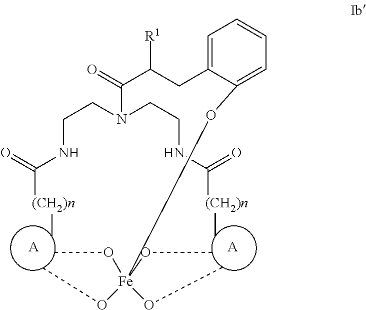

or a salt thereof,
wherein n is 0 or 1; R¹ is H or —W^{b}.

In one embodiment the compound of formula I is:

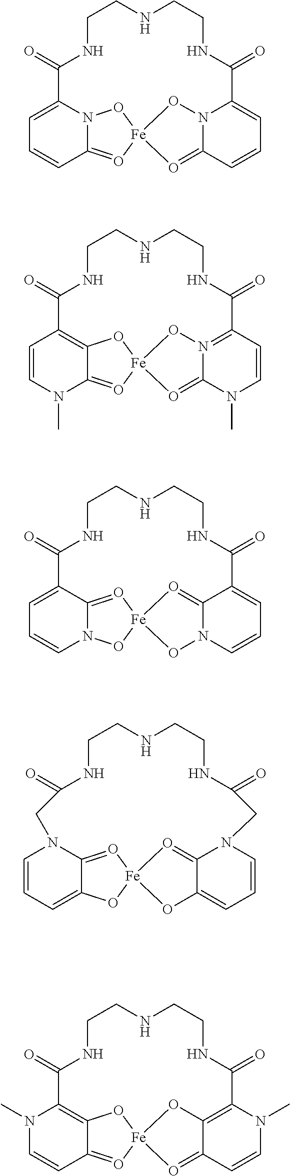

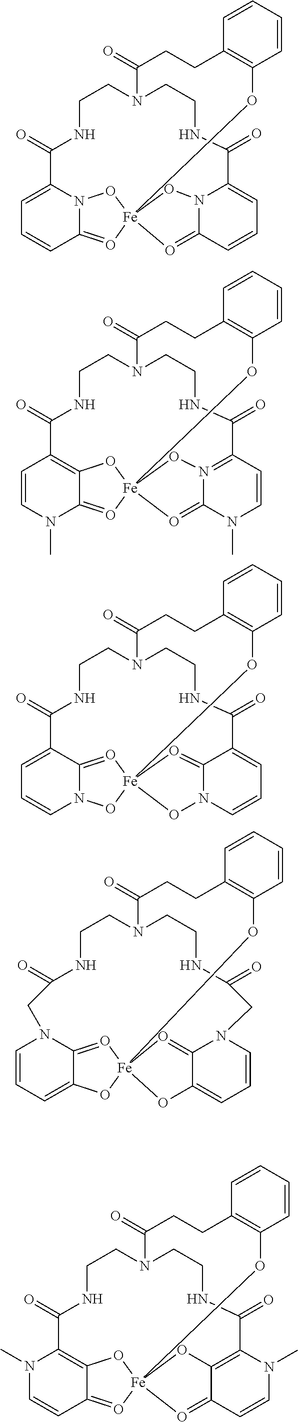

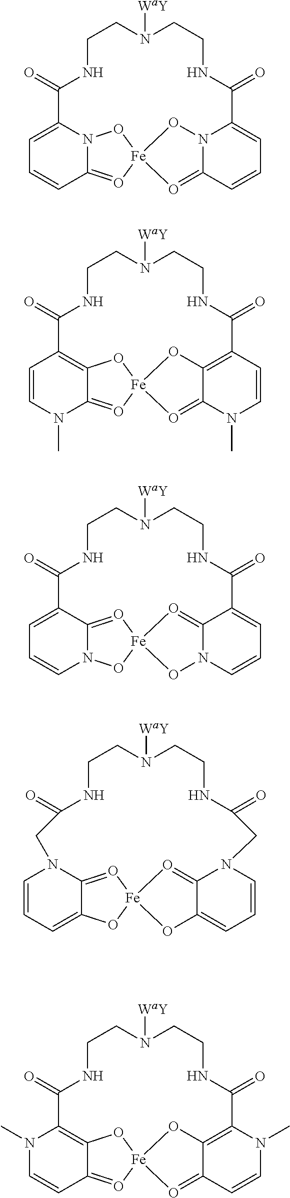

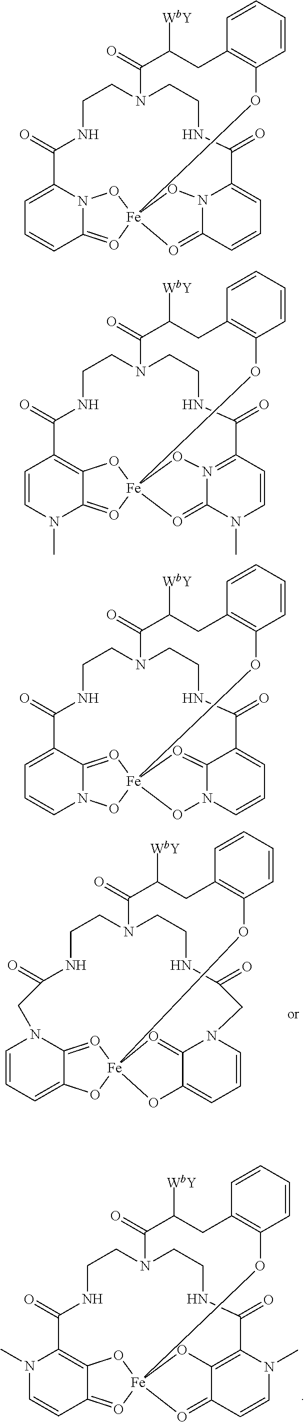

In one embodiment the compound of formula I is:

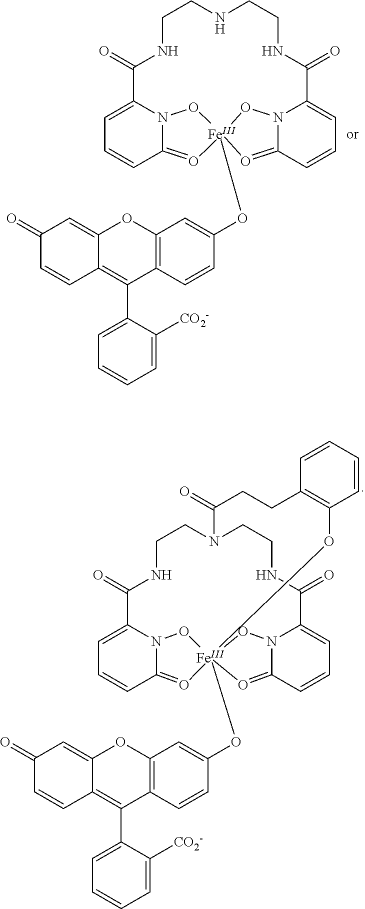

One embodiment provides a material or device comprising one or more iron complexes or salts thereof as described herein. Ine one embodiment the material or device is attached to the one or more iron complexes or salts thereof at the linker W^{a} or W^{b}.

One embodiment provides a material or device comprising one or more iron complexes selected from

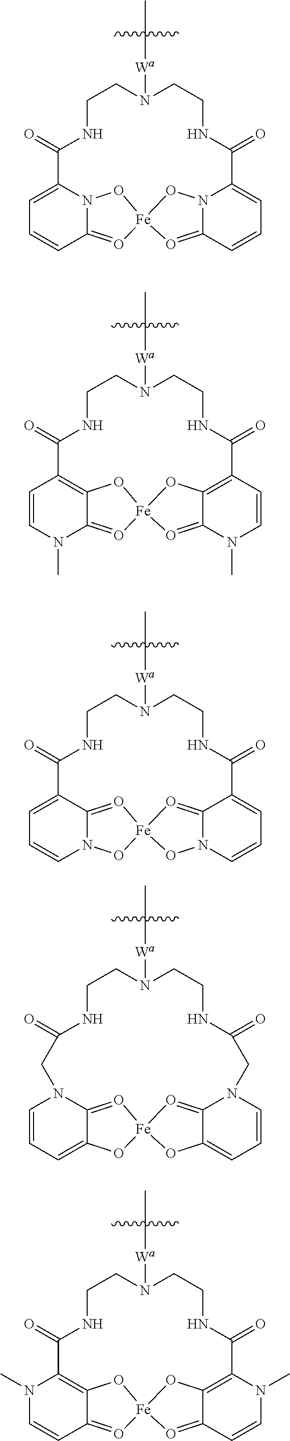

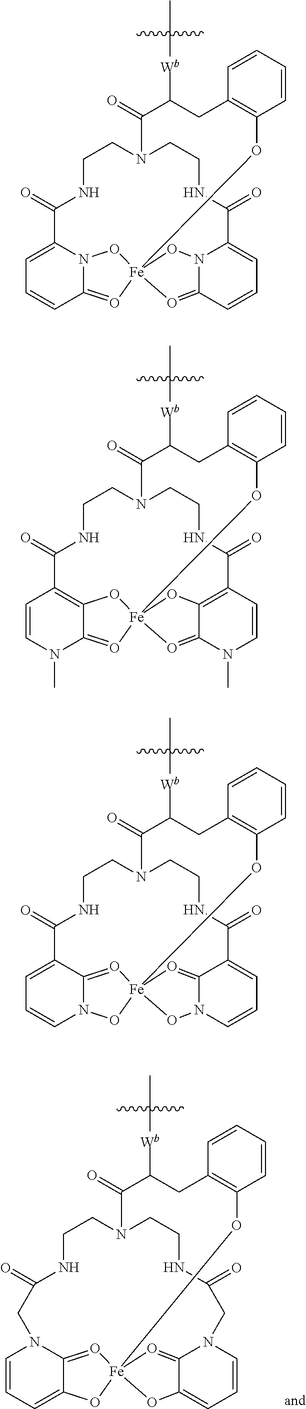

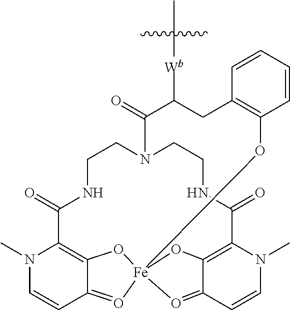

or a salt thereof, wherein the material or device is bonded to W^{a} or W^{b} as shown by the wavy line.

In one embodiment
W^{a} is

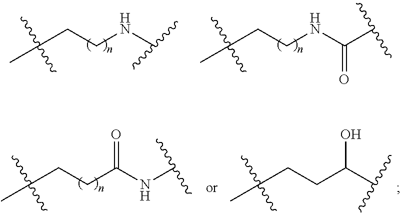

W^{b} is

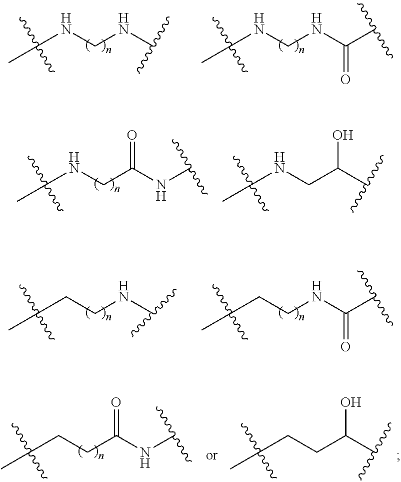

and
n is 1, 2, 3, 4, 5, 6, 7, 8, 9, or 10.

One embodiment provides a ligand or salt thereof of formula II

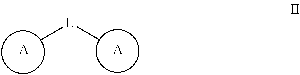

wherein:
each

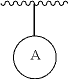

moiety is independently a pyridinone substituted with one hydroxy or —O⁻ wherein the pyridinone is optionally substituted with one or more (C₁-C₆)alkyl, and wherein each dashed bond is independently a single or a double bond;
L is a linker, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y;
X^{a} is phenyl substituted with hydroxy or —O—;
W^{a} is a linker^{a} group;
W^{b} is a linker^{b} group; and
Y is a polymer, hydrogel, membrane, nanoparticle, or material.

One embodiment provides the ligand of formula II

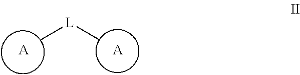

wherein:
each

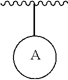

moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.
X^{a} is phenyl substituted with hydroxy or —O⁻;
W^{a} is a linker^{a} group; and
W^{b} is a linker^{b} group.

One embodiment provides the ligand of formula II, wherein A, L, X^{a}, W^{a}, W^{b} and Y are as defined in any embodiment or claim provided herein.

One embodiment provides the

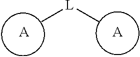

compound as described herein that does not include the iron atom.

One embodiment provides an iron complex comprising a compound of formula Ic

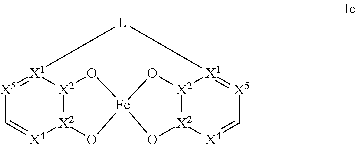

or a salt thereof,
wherein Fe is Fe^{II} or Fe^{III} or a combination thereof;
X¹ is C orN;
X² together with oxygen to which it is attached is N—O, C—O, C═O;
X³ together with oxygen to which it is attached is N—O, C—O, C═O;
X⁴ is C or NRa;
X⁵ is C or NRa
wherein the ring formed by X¹, X², X³, X⁴, X⁵ and the carbon is a pyridinone, wherein the pyrdinone is substituted with one hydroxy or —O, wherein the pyridinone is optionally substituted with one or more (C₁-C₆)alkyl;
each R^{a} is independently (C₁-C₆)alkyl.
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.
X^{a} is phenyl substituted with hydroxy or —O—;
W^{a} is a linker^{a} group; and
W^{b} is a linker^{b} group.

One embodiment provides an iron complex comprising a compound of formula Id

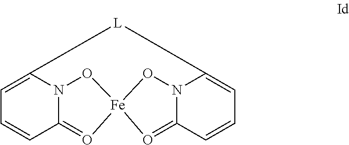

or a salt thereof.

One embodiment provides an iron complex as described herein further comprising a weak binding ligand. In one embodiment the weak binding ligand is fluorescein.

One embodiment provides a method to detect inorganic phosphate comprising contacting the phosphate with an iron complex as described herein. In one embodiment the phosphate is selectively detected in the presence of other anions. In one embodiment the anions are selected from the group consisting of carbonate, nitrate, sulfate, halides, arsenate and pyrophosphate. Ine one embodiment the phosphate is contacted with the iron complex as a liquid sample at about neutral pH. In one embodiment the liquid sample is obtained from a body of water. In one embodiment the liquid sample is a eutrophic sample. In one embodiment the phosphate is detected by fluorescence sensing by an indicator displacement assay.

One embodiment provides a method to remove inorganic phosphate from an aqueous mixture or solution comprising contacting the aqueous mixture or solution with an iron complex as described herein. One embodiment provides a method to sequester or remove inorganic phosphate from an aqueous mixture or solution comprising contacting the aqueous mixture or solution with an iron complex as described herein. One embodiment provides a method to remove inorganic phosphate from an aqueous mixture or solution comprising contacting the aqueous mixture or solution with an iron complex as described herein, under conditions wherein the phosphate binds to the iron complex and is partially or completely removed from the mixture or solution. One embodiment provides a method to sequester or remove inorganic phosphate from an aqueous mixture or solution comprising contacting the aqueous mixture or solution with an iron complex as described herein, under conditions wherein the phosphate binds to the iron complex and is partially or completely removed from the mixture or solution..

The invention will now be illustrated by the following non-limiting example.

### Example 1

### Experimental Section

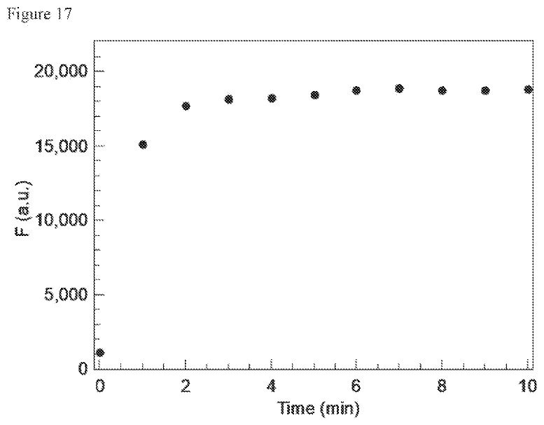

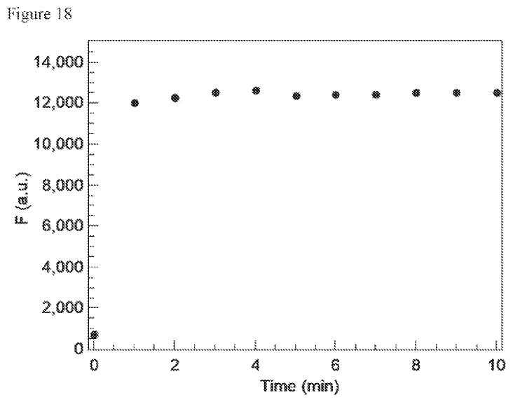

Unless otherwise stated, all chemicals were purchased from commercial suppliers and used without further purification. Deuterated solvents were obtained from Cambridge Isotope Laboratories (Tewskbury, MA, USA). Distilled water was further purified by a Millipore Simplicity UV system (resistivity 18×10⁶Ω). All organic extracts were dried over anhydrous MgSO₄ (s). NaF, NaCl, NaBr, NaI, Na₂SO₄, NaNO₃, NaHCO₃, NaOAc, Na₄P₂O₇, and Na₂HAsO_{4·}7H₂O were used for anion screen studies. Flash chromatography was performed on Merck Silica Gel. Modified silica gel was prepared by heating the silica gel in 37% HCl (aq) at 50° C. for 6 h, further washing it with deionized water until the pH of the filtrate was neutral and drying it under reduced pressure at 100° C. The collected silica gel was subsequently suspended in toluene with 1% (v/v) hexadecyltrimethoxysilane. The mixture was stirred at 100° C. for 6 h, after which the mixture was filtered, rinsed with toluene and ethyl acetate, and dried under reduced pressure at 100° C. ¹H NMR and ¹³C NMR spectra were recorded at room temperature on a Bruker Advance III 400 at 400 MHz and 100 MHz, respectively, or a Bruker Advance III AV 500 at 500 MHz and 125 MHz, respectively, at the LeClaire-Dow instrumentation facility of the Department of Chemistry of the University of Minnesota. The residual solvent peaks were used as internal references. Data for ¹H NMR are recorded as follows: chemical shift (δ, ppm), multiplicity (s, singlet; d, doublet, t, triplet; q, quartet; br, broad; m, multiplet), coupling constant (Hz), integration. Data for ¹³C NMR are recorded as follows: chemical shift (δ, ppm). Low resolution (LR) and high resolution (HR) electrospray spray ionization time-of-flight mass spectrometry (ESI/TOF-MS) were recorded on a Bruker BioTOF I at the LeClaire-Dow instrumentation facility of the Department of Chemistry of the University of Minnesota. UV-visible spectra were recorded on a Varian Cary 100 Bio Spectrophotometer. Data was collected over the range of 200-800 nm. Luminescence data was acquired on a Varian Cary Eclipse Fluorescence Spectrophotometer using a quartz cell with a path length of 1 cm and chamber volume 400 μL. Sample solutions were allowed to equilibrate for 5 min before measurement of their luminescence spectra, as initial studies demonstrated that this time was sufficient to achieve thermodynamic equilibrium (FIG. 17 and FIG. 18). All fluorescent titration data were acquired at room temperature (T=25° C.). Every data point was measured in triplicate from three independently prepared samples. For fluorescent titrations or anion selectivity studies, 5 mM of anions were prepared in water and pH adjusted to 7 using 0.1 N HCl or 0.1 N NaOH. Wet ethanol denotes the water content is ≤10 (v/v %) in ethanol. Luminescence data were processed with Scilab 6.0.2 and QtiPlot 0.9.8.9 software. All pH measurements were performed using Thermo Scientific Ag/AgCI refillable probe and a Thermo Orion 3 Benchtop pH meter. High-performance liquid chromatography (HPLC) data was collected on a Varian Prostar Model 210, coupled with an Agilent ZORBAX Eclipse XDB-C18 column, and a Varian ProStar 335 diode array detector. Unless specified otherwise HPLC measurements were performed at a flow rate 1.0 mL min⁻¹ with the following elution condition: 15% CH₃CN/85% water from 0 to 10 minutes, followed with a linear gradient to 85% CH₃CN/15% water from 10 to 23 minutes, 85% CH₃CN/15% water from 23 to 45 minutes.

### Synthesis

4-Nitrophenyl 1-(benzyloxy)-6-oxo-1,6-dihydropyridine-2-carboxylate (3). Thep-nitrophenol activated ester was prepared in reference to a similar procedure for the preparation of N-hydroxysuccinimidyl ester of the podant (F. Guérard, M. Beyler, Y.-S. Lee, R. Tripier, J.-F. Gestin, M. W. Brechbiel, Dalton Trans. 2017, 46, 4749-4758). HOPO(Bn)-OH (1000. mg, 4.080 mmol) was suspended in anhydrous methylene chloride (10 mL) under N₂ atmosphere, followed by the injection of oxalyl chloride (400. μL, 4.90 mmol) and one drop of DMF. The reaction was let stir for 1 hr at room temperature before the removal of solvent, HCl, and excess oxalyl chloride by high vacuum with liquid N₂ trap. Under N₂ atmosphere, the residue was dissolved in 10 mL anhydrous methylene chloride. After p-nitrophenol (568 mg, 4.08 mmol) was added to the solution, the mixture was cooled by ice bath, and NEt₃ (900 μL, 6.10 mmol) was injected slowly to the mixture. The mixture was then warmed up to room temperature for 6 hr, after which it was washed with 10% citric acid solution, 10% sodium bicarbonate solution, dried over anhydrous MgSO₄ (s), and concentrated under vacuum to a viscous liquid. This crude product was purified by flash chromatography over silica eluting with 60% EtOAc/20% Hex to yield white solid 3 (1.363 g, 91%). ¹H NMR (400 MHz, CDCl₃): δ 8.29 (d, J=9 Hz, 2H), 7.49 (d, J=6 Hz, 2H), 7.41-7.26 (m, 6H), 6.94 (d, J=9 Hz, 1H), 6.79 (d, J=7 Hz, 1H), 5.42 (s, 2H). ¹³C NMR (100 MHz, CDCl₃): δ 158.6, 157.2, 154.4, 145.8, 137.1, 136.9, 133.3, 130.2, 129.4, 128.6, 127.6, 125.3, 122.2, 109.4, 78.8. ESI-HRMS: m/z=389.0755 ([M+Na]⁺), (Calcd. 389.0744).

N,N′-(Azanediylbis(ethane-2,1-diyl))bis(1-(benzyloxy)-6-oxo-1,6-dihydropyridine-2-carboxamide) (4). p-nitrophenol activated ester 3 (808 mg, 2.207 mmol) was dissolved in 20 mL methylene chloride, followed by slow injection of diethylenetriamine (119 μL, 1.10 mmol) and NEt₃ (300. μL, 2.21 mmol). The reaction was stirred at room temperature for 1 hr. The mixture was washed with 20 mL of 1N NaOH solution, dried over anhydrous MgSO₄ (s), and concentrated under vacuum to a viscous liquid. This crude product was purified by flash chromatography over silica eluting with 12% MeOH/88% CH₂Cl₂ to yield foaming liquid 4 (1.045 g, 85%). ¹H NMR (400 MHz, CDCl₃): δ 7.59 (br, 2H), 7.35-7.22 (m, 10H), 7.14 (dd, J₁=9 Hz, J₂=7 Hz, 2H), 6.36 (d, J=9 Hz, 2H), 6.21 (d, J=7 Hz, 2H), 5.18 (s, 4H), 3.14 (d, J=10 Hz, 4H), 2.51-2.48 (m, 4H), 1.62 (br, 1H). ¹³C NMR (100 MHz, CDCl₃): δ 160.4, 158.3, 143.0, 138.3, 133.3, 129.6, 129.0, 128.3, 123.0, 105.6, 78.9, 47.1, 39.3. ESI-HRMS: m/z=580.2156 ([M+Na]⁺), (Calcd. 580.2167).

N,N-(Azanediylbis(ethane-2,1-diyl))bis(1-hydroxy-6-oxo-1,6-dihydropyridine-2-carboxamide) HBr (5). The protected ligand 4 (200. mg, 0.359 mmol) was dissolved in 1 mL of glacial acetic acid, and 1 mL of 30% HBr was added to the reaction mixture. After 6 hr, acetic acid and HBr were removed under high vacuum to yield a foaming liquid. This crude product was purified by flash chromatography over modified silica eluting with 0% MeOH/100% H₂O gradient to 5% MeOH/95% H₂O to yield white foaming liquid 5 (150 mg, 91%). ¹H NMR (400 MHz, CF₃COOD): δ 7.80 (t, J=8 Hz, 2H), 7.52 (d, J=8 Hz, 2H), 7.30 (d, J=9 Hz, 2H), 4.00 (br, 4H), 3.65 (br, 4H). ¹³C NMR (100 MHz, CF₃COOD): δ 165.4, 160.1, 141.4, 138.0, 121.9, 118.6, 51.6, 40.0. ESI-HRMS: m/z=378.1310 ([M-Br]⁺), (Calcd. 378.1408).

Fe^{III}—HOPO-fluo (1). Ligand 5 (5.0 mg, 0.011 mmol) and fluorescein (3.6 mg, 0.011 mmol) was suspended in anhydrous EtOH (10 mL), followed by injection of 1N NaOH (22 μL, 0.022 mmol). 0.1 N ethanolic FeBr₃ (110 μL, 0.011 mmol) was then added to the reaction mixture. The purity and identity of Fe^{III}—HOPO-fluo formed in situ were characterized by HPLC and ESI-HRMS. The solution was used without further purification. ESI-HRMS: m/z=763.1138 ([M-Br]⁺), (Calcd. 763.1208).

3-(2-(Benzyloxy)phenyl)propanoic acid (6). The benzyl protected side arm 6 was synthesized according to the reported procedure^{[2]} and characterized by NMR and ESI-HRMS. ¹H NMR (400 MHz, CDCl₃): δ 11.05 (br, 1H), 7.46-7.32 (m, 5H), 7.26-7.19 (m, 2H), 6.94-6.90 (m, 2H), 5.12 (s, 2H), 3.04 (t, J=8 Hz, 2H), 2.72 (t, J=8 Hz, 2H). ¹³C NMR (100 MHz, CDCl₃): δ 179.6, 156.5, 137.2, 130.1, 128.8, 128.6, 127.8, 127.7, 127.0, 120.8, 111.6, 69.8, 34.0, 25.9. ESI-HRMS: m/z=279.1009 ([M+Na]⁺), (Calcd. 279.0992).

1-(Benzyloxy)-N-(2-(N-(2-(1-(benzyloxy)-6-oxo-1,6-dihydropyridine-2-carboxamido)ethyl)-3-(2-(benzyloxy)phenyl)propanamido)ethyl)-6-oxo-1,6-dihydropyridine-2-carboxamide (7). The protected side arm 6 (316 mg, 1.23 mmol) was suspended in anhydrous methylene chloride (10 mL) under N₂ atmosphere, followed by the injection of oxalyl chloride (0.1 mL, 1.4 mmol) and one drop of DMF. The reaction was let stir for 1 hr at room temperature before the removal of solvent, HCl, and excess oxalyl chloride by high vacuum with liquid N₂ trap. Under N₂ atmosphere, the residue was dissolved in 10 mL anhydrous methylene chloride and cooled with ice bath. The protected ligand 4 (687 mg, 1.23 mmol) was dissolved in anhydrous methylene chloride (5 mL) and slowly injected to the cooled reaction mixture. To the mixture was then injected NEt₃ (344 μL, 2.47 mmol). The mixture was then warmed up to room temperature for 1 hr, after which it was washed with 10% citric acid solution, dried over anhydrous MgSO₄ (s), and concentrated under vacuum to a viscous liquid. This crude product was purified by flash chromatography over silica eluting with 4% MeOH/96% CH₂Cl₂ to yield white foaming liquid 7 (805 mg, 82%). ¹H NMR (400 MHz, CDCl₃): δ 7.48-7.46 (m, 2H), 7.41-7.36 (m, 5H), 7.34-7.27 (m, 9H), 7.25-7.21 (m, 2H), 7.13-7.09 (m, 2H), 6.94 (br, 1H), 6.89-6.82 (m, 3H), 6.68 (dd, J₁=1 Hz, J₂=8 Hz, 2H), 6.24 (t, J=2 Hz, 1H), 6.23 (t, J=2 Hz, 1H), 5.32 (s, 2H), 5.24 (s, 2H), 5.02 (s, 2H), 3.31 (br, 4H), 3.06 (br, 4H), 2.88 (t, J=7 Hz, 2H), 2.48 (t, J=8 Hz, 2H). ¹³C NMR (100 MHz, CDCl₃): δ 174.3, 160.7, 160.6, 158.5, 158.4, 156.5, 142.8, 142.2, 138.0, 137.9, 137.0, 133.5, 133.1, 130.3, 130.1, 129.5, 129.1, 128.60, 128.55, 128.50, 128.0, 127.7, 127.3, 124.1, 120.8, 111.6, 105.9, 105.2, 79.3, 79.0, 69.9, 47.40, 45.7, 39.3, 38.7, 32.8, 26.9. ESI-HRMS: m/z=818.3181 ([M+Na]⁺), (Calcd. 818.3160).

1-Hydroxy-N-(2-(N-(2-(1-hydroxy-6-oxo-1,6-dihydropyridine-2-carboxamido)ethyl)-3-(2-hydroxyphenyl)propanamido)ethyl)-6-oxo-1,6-dihydropyridine-2-carboxamide (8). The protected ligand 7 (450. mg, 0.566 mmol) was dissolved in 1 mL of glacial acetic acid, and 1 mL of 30% HBr was added to the reaction mixture. After 6 hr, acetic acid and HBr were removed under high vacuum to yield a foaming liquid. This crude product was purified by flash chromatography over modified silica eluting with 0% MeOH/100% H₂O gradient to 5% MeOH/95% H₂O to yield white foaming liquid 8 (270 mg, 91%). ¹H NMR (500 MHz, CD₃OD): δ 7.48-7.42 (m, 2H), 7.04 (d, J=7 Hz, 1H), 6.97 (dd, J₁=1 Hz, J₂=8 Hz, 2H), 6.75-6.68 (m, 4H), 6.61 (dd, J₁=1 Hz, J₂=7 Hz, 1H), 6.56 (dd, J₁=1 Hz, J₂=7 Hz, 1H), 3.65-3.61 (m, 4H), 3.56-3.54 (m, 4H), 2.88 (t, J=7 Hz, 2H), 2.75 (t, J=8 Hz, 2H). ¹³C NMR (125 MHz, CD₃OD): δ 176.5, 162.8, 162.7, 160.2, 160.1, 156.5, 142.0, 141.7, 139.0, 138.8, 131.5, 128.7, 120.94, 120.85, 116.4, 108.7, 108.5, 48.3, 46.3, 39.4, 39.0, 34.7, 27.6. ESI-HRMS: m/z=524.1775 ([M−H]⁻), (Calcd. 524.1776).

Fe^{III}—HOPO-PhO-fluo (2). Ligand 8 (5.0 mg, 9.5 μmol) and fluorescein (3.6 mg, 9.5 μmol) was suspended in anhydrous EtOH (10 mL), followed by injection of 1N NaOH (29 μL, 29 μmol). 0.1 N ethanolic FeBr₃ (95 μL, 9.5 μmol) was then added to the reaction mixture. The purity and identity of Fe^{III}—HOPO-PhO-fluo formed in situ were characterized by HPLC and ESI-HRMS. The solution was used without further purification. ESI-HRMS: m/z=911.1830 ([M+3H]⁺), (Calcd. 911.1733).

### Data Fitting

Data fitting of Fe^{III}-complexes+Pi uses the following equations:

$$[ H ] + [ G ] ⇌ [ HG ]$$

where H denotes the host (Fe^{III}-complex-fluo), G the guest (Pi), and HG the adduct. Equilibrium constants K is defined as:

$$K = \frac{[ HG ]}{[ H ] [ G ]}$$

The luminescence intertity increase can be described as the following:²

$$F - {F}_{0} = \frac{1}{{2 [ H ]}_{0}} Δ {F}_{Max} \{ {[ G ]}_{0} + {[ H ]}_{0} + \frac{1}{K} - \sqrt{{( {[ G ]}_{0} + {[ H ]}_{0} + \frac{1}{K} )}^{2} - {{4 [ G ]}_{0} [ H ]}_{0} \}}$$

where
F=integrated fluorescence,
F₀=initial integrated fluorescence,

$$Δ {F}_{max} = {F}_{∞} - {F}_{0} .$$

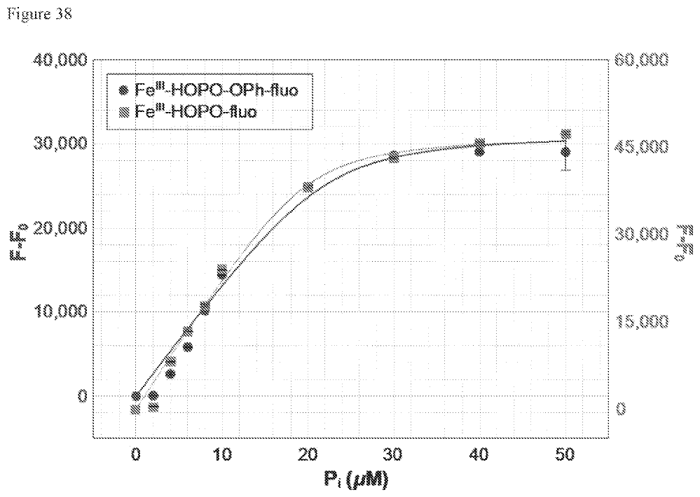

Non-linear fitting was performed using QtiPlot 1.0.0 software.
Detection limits were calculated according to 3σ, where σ is the standard deviation of the fitting.
   The fitting results are shown in FIG. 38 and Table S1

TABLE S1
Fitting results of Fe^{III}—HOPO-fluo + Pi and
Fe^{III}—HOPO—OPh-fluo + Pi.

| | K (M⁻¹) | R² | σ |
|---|---|---|---|
| Fe^{III}—HOPO—OPh-fluo | 8.8 ± 3.4 × 10⁵ | 0.982 | 1665 |
| Fe^{III}—HOPO-fluo | 1.1 ± 0.5 × 10⁶ | 0.992 | 1737 |

### DISCUSSION

The receptors Fe^{III}—HOPO-fluo and Fe^{III}—HOPO-PhO-fluo were synthesized according to Schemes 1 and 2, respectively. The p-nitrophenol activated ester of the benzyl-protected HOPO podand 3, previously synthesized following literature precedence,³⁹ selectively acylate the primary amino groups of the triamine backbone to yield the protected ligand 4. Deprotection under strong acidic conditions yields the final ligand 5, which was further metallated with Fe^{III} in the presence of fluorescein to give the final receptor Fe^{III}—HOPO-fluo.

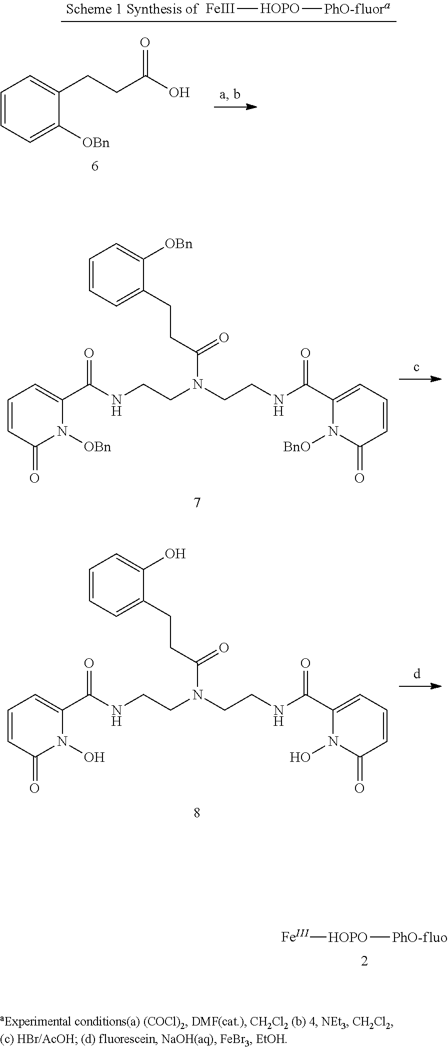

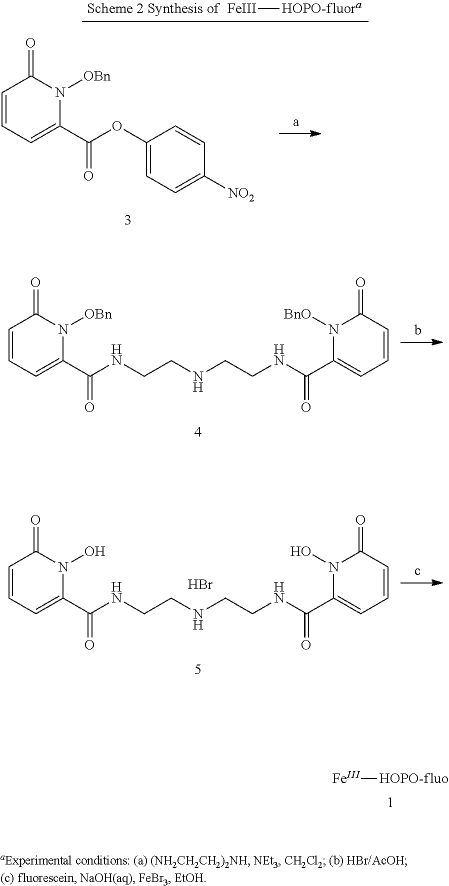

Fe^{III}—HOPO-PhO-fluo employs a pentadentate ligand whose phenolate moiety occupies one more coordination site of the Fe^{III} center. Activation of the benzyl-protected phenol podand 6, previously synthesized according to literature reports,⁴⁰ with oxalyl chloride enabled coupling to the central secondary amine of 4, thereby yielding the protected ligand 7. Deprotection under strong acidic conditions yielded the final ligand 8 that was further metallated with Fe^{III} in the presence of fluorescein to give the final receptor Fe^{III}—HOPO-PhO-fluo, 2.

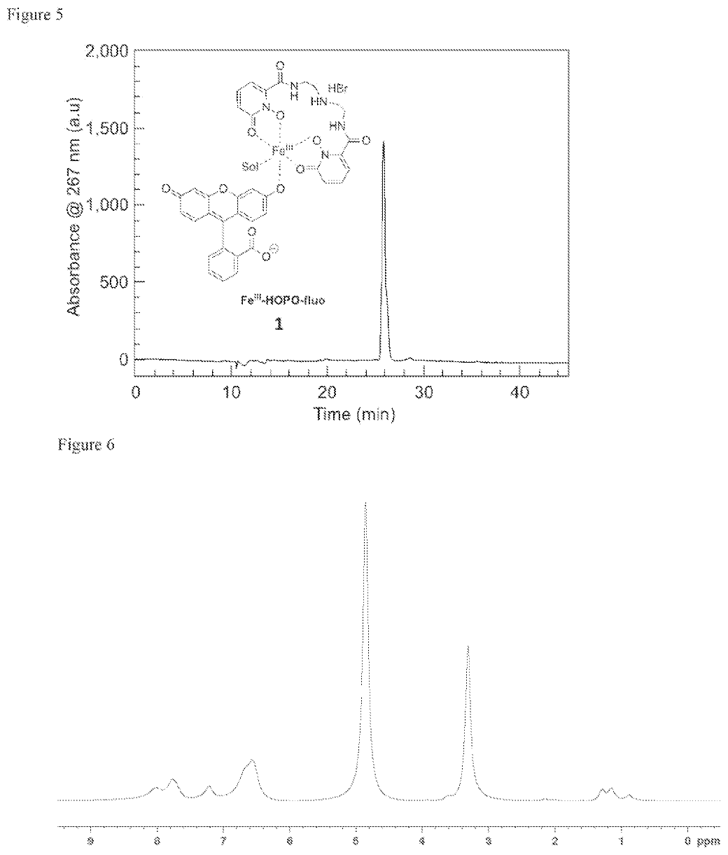

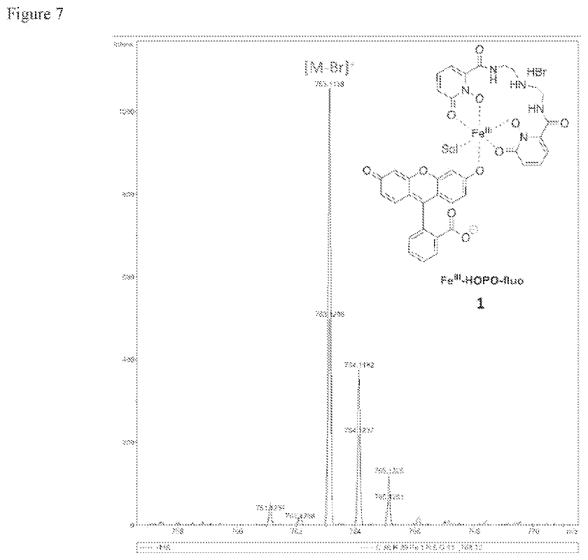

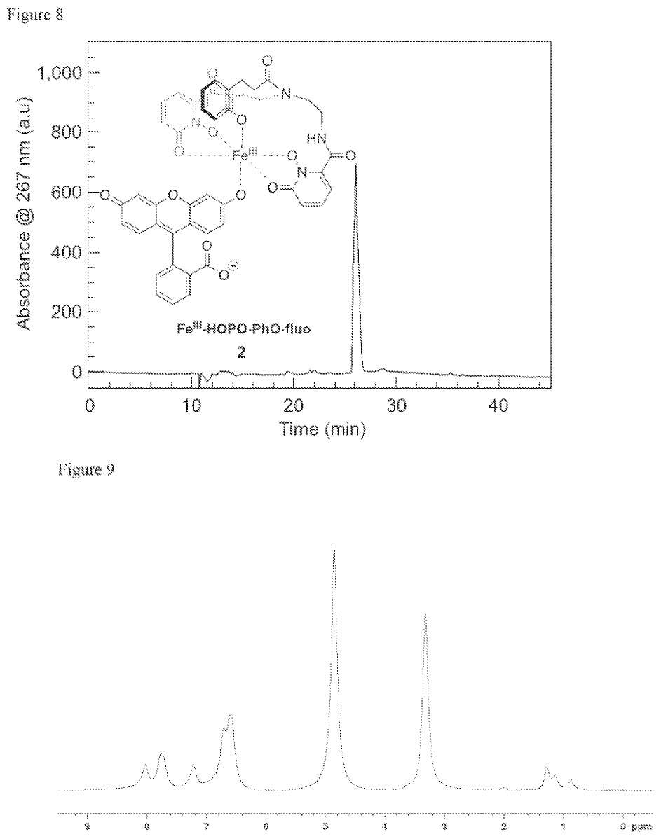

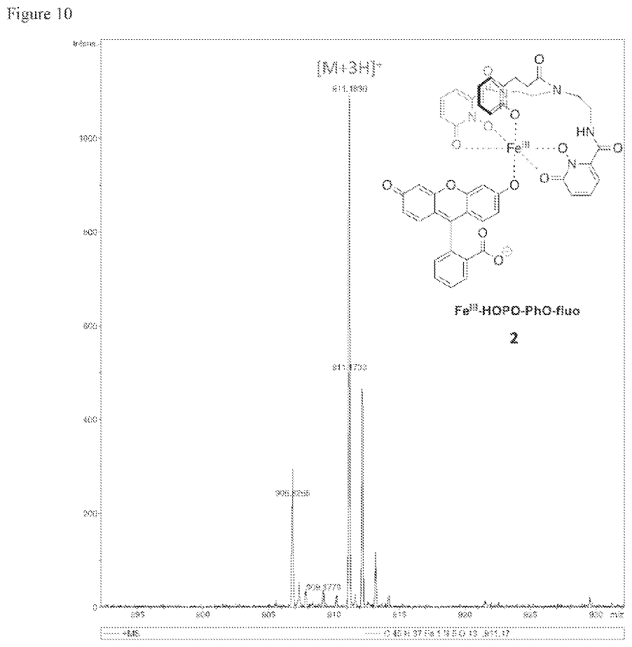

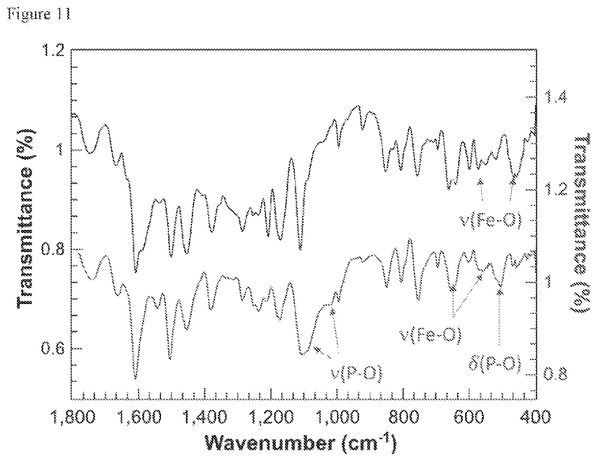

In both syntheses, formation and purity of the ternary complexes 1 and 2 was confirmed by HPLC and ESI-MS (FIGS. 5, 7, 8, and 10). No μ-oxo diiron dimer were detected, confirming that coordination of the fluorescein ligand is sufficient to protect the Fe^{III} center and prevent the formation of bimetallic species. In contrast, in the absence of fluorescein, the μ-oxo diiron dimer is the predominant species observed by MS. The significant line broadening observed in the ¹H NMR of the ternary complexes in solution (FIGS. 6 and 9), which is typical of paramagnetic Fe(III) species, further confirmed coordination of fluorescein to the receptors 1 and 2. Both Fe^{III}·fluorescein complexes were stable as solids and in ethanol for weeks; both can tolerate up to 10 vol % water with pH adjusted to 7 without significant fluorescein dissociation (<1%) in ethanol.

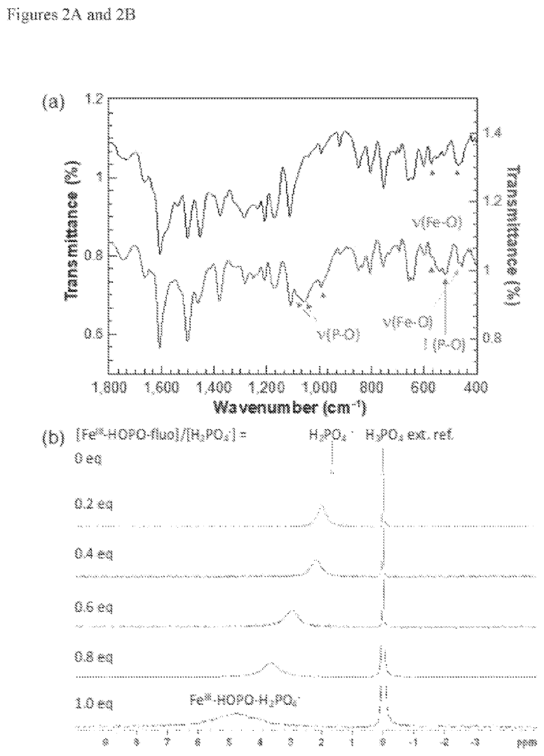

Direct coordination of phosphate to the iron centers of the receptors concomitant with displacement of the fluorescein moiety upon addition of the oxyanion was first confirmed from attenuated total reflection-infrared (ATR-IR) spectroscopic analysis of the precipitate obtained from Fe^{III}—HOPO-fluo+Pi and Fe^{III}—HOPO-PhO-fluo+Pi. The iron complex Fe^{III}—HOPO-Pi displays the characteristic v(Fe—O) vibrations at 571 and 461 cm⁻¹, v(P—O) bands at 1088, 1067, 968 cm⁻¹ and δ (O—P—O) bands at (541) cm⁻¹ (FIG. 2A).⁴¹⁻⁴⁴ Each of those bands was also observed for the Fe^{III}—HOPO-PhO-Pi adduct (FIG. 11). These observations are in agreement with the formation of the postulated ternary complexes.

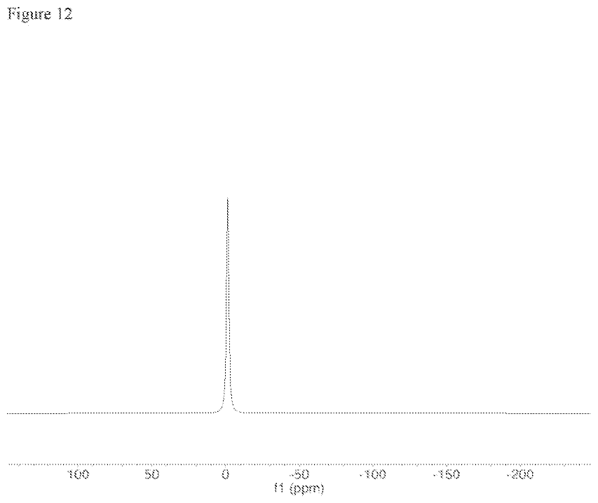

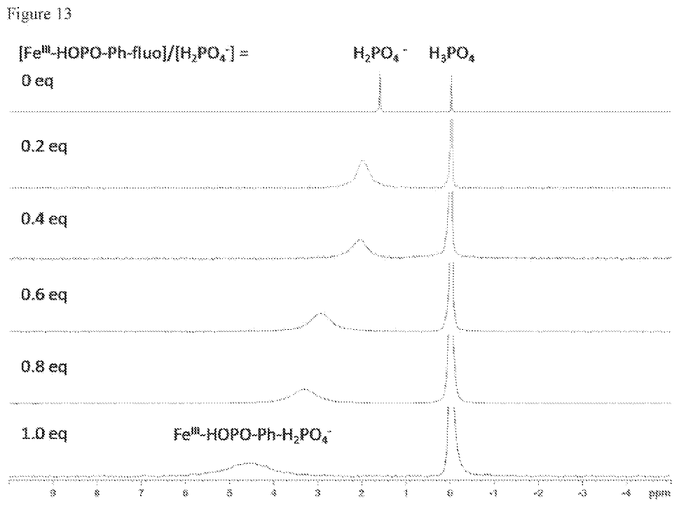

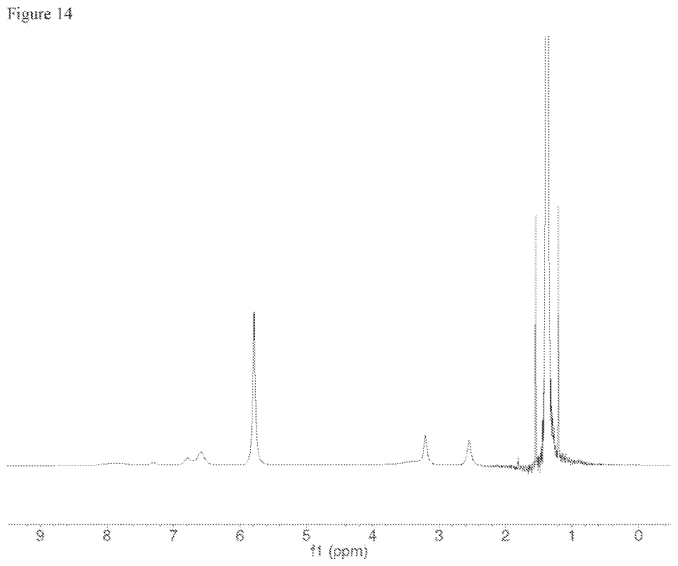

Formation of a Fe^{III}L·Pi ternary complex was also supported by NMR spectroscopy. The ³¹P NMR spectrum of Fe^{III}—HOPO-Pi is nearly featureless (FIG. 12), an observation that is attributed to the shortened transverse relaxation times, T₂, of the ³¹P nucleus by the strongly paramagnetic Fe^{III}. As is apparent in FIG. 2B, when referenced to an external standard of H₃PO₄, in a titration monitored by NMR, the ³¹P signal of phosphate progressively shifts downfield from 1.61 to 4.72 ppm upon gradual addition of Fe^{III}—HOPO-fluo (1). This shift is accompanied by a significant line broadening corresponding to a decrease in T₂ of the phosphorus nuclei from 0.11 s (no Fe^{III}—HOPO-fluo) to 1.95 ms (1 equivalent of Fe^{III}—HOPO-fluo). Both of those observations are attributable to coordination of orthophosphate to the strongly paramagnetic Fe^{III} center.^{45,46} Of note, the presence of a single peak in the ³¹P also suggests the presence of a rapid equilibrium between bound and free phosphate. Fe^{III}—HOPO-PhO-fluo (2), which employs a pentadentate ligand, displays similar behavior with the coordination of phosphate to the Fe^{III} center confirmed from both the ATR-IR and the ³¹P NMR spectra (FIGS. 11 and 12, respectively). Unfortunately, further attempts to characterize the ternary phosphate complexes by mass spectrometry were unsuccessful due to the their low solubility and the known ability of phosphate to suppress ionization.^{47,48}

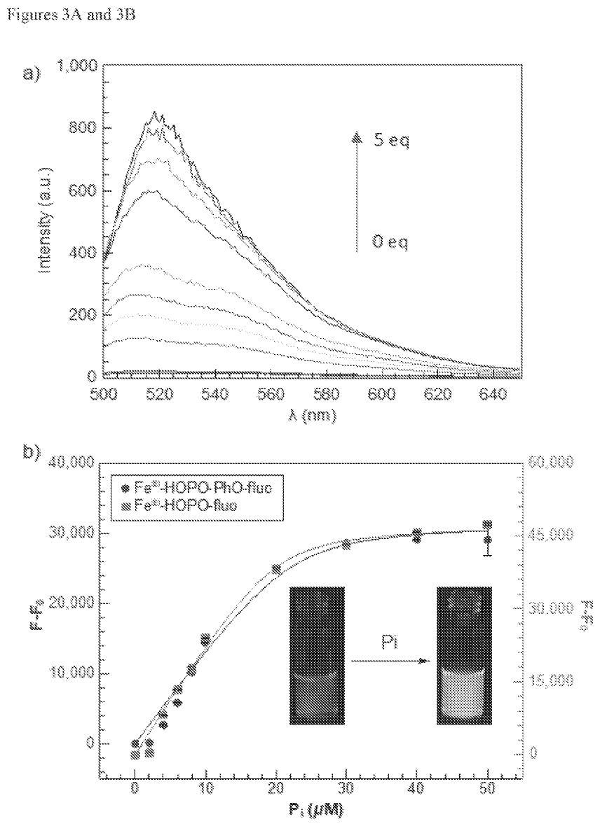

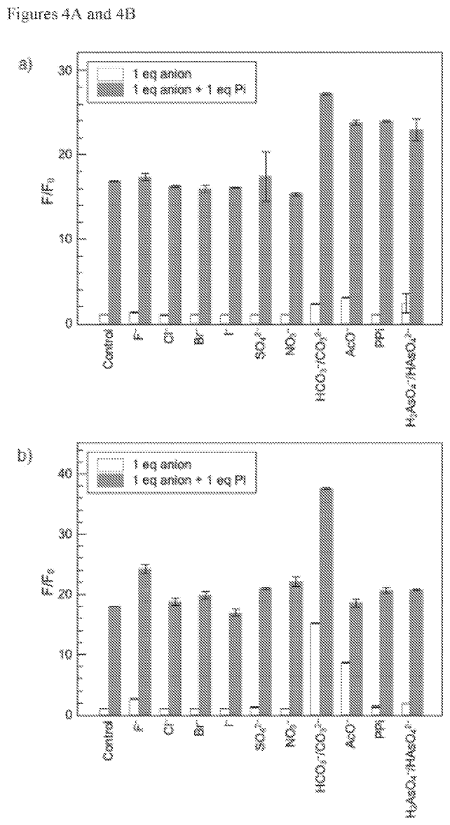

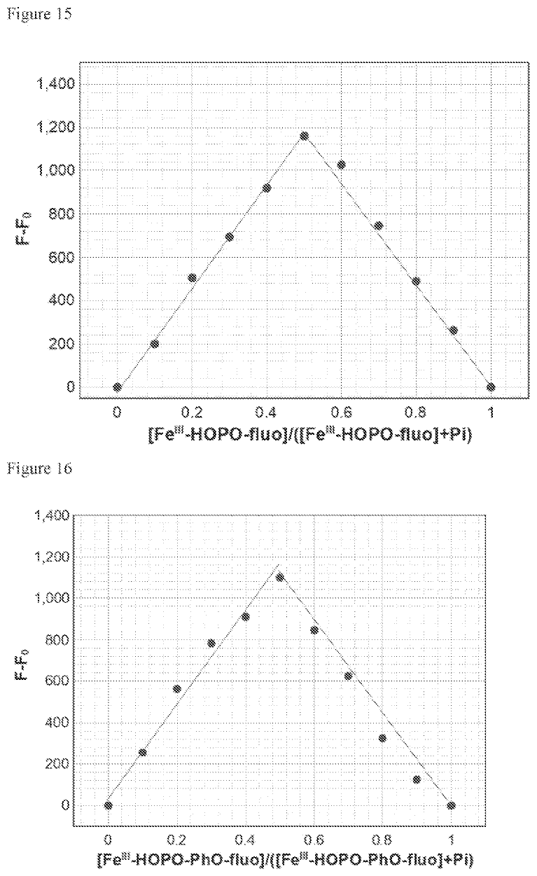

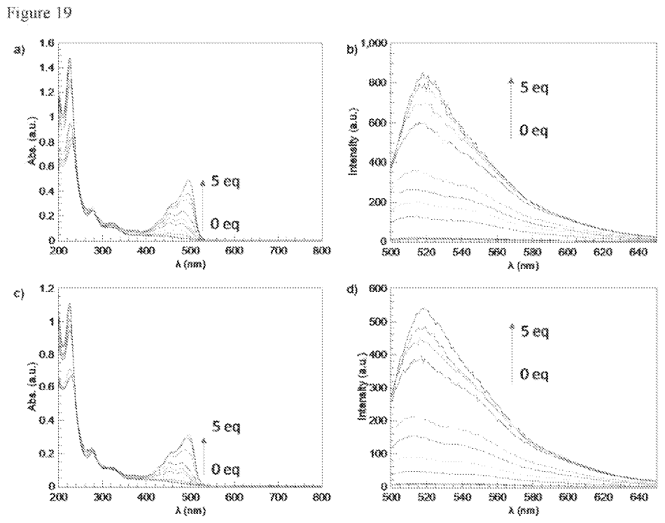

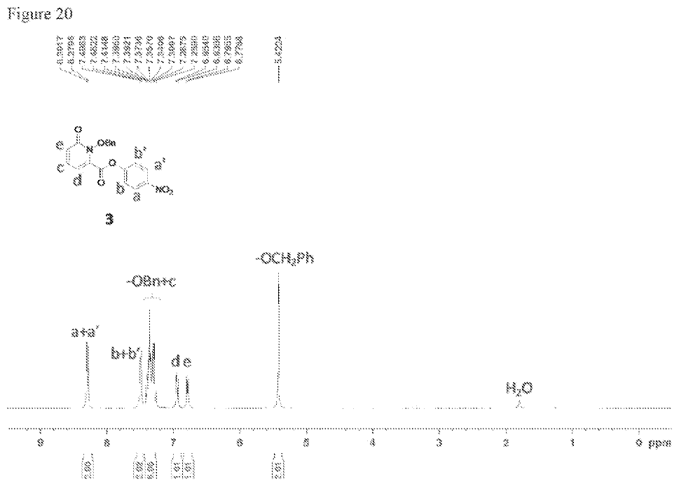

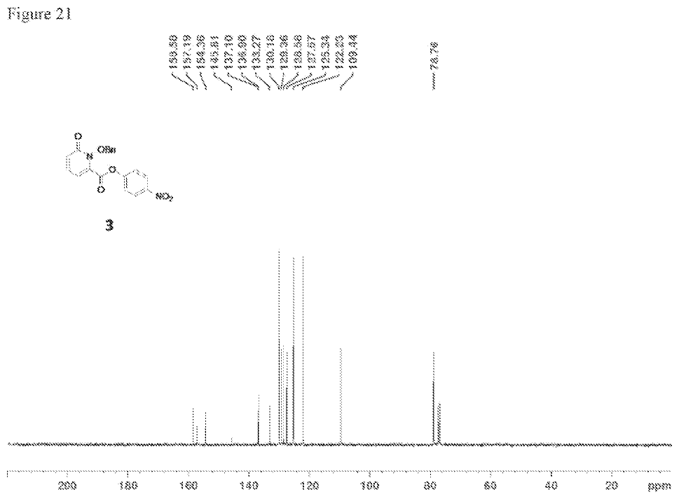

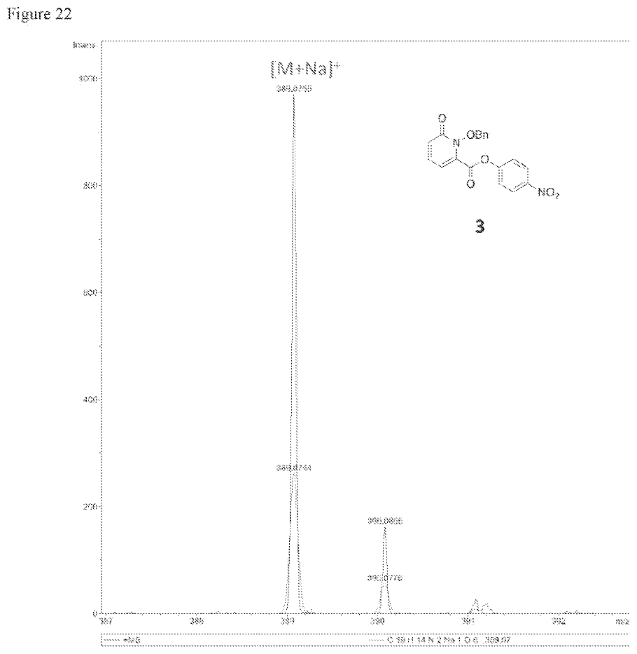

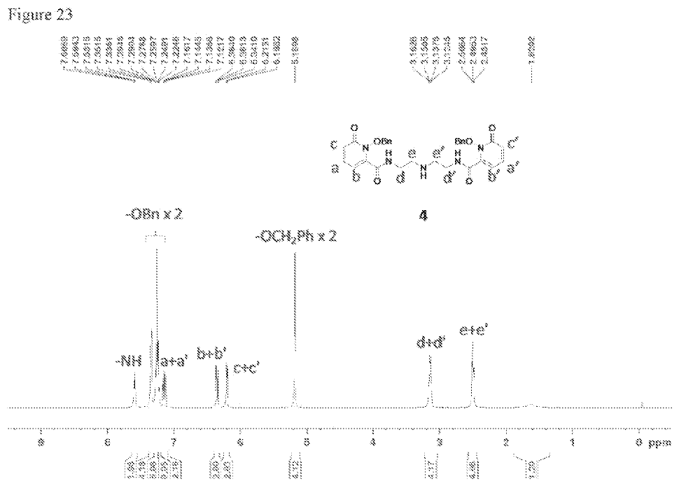

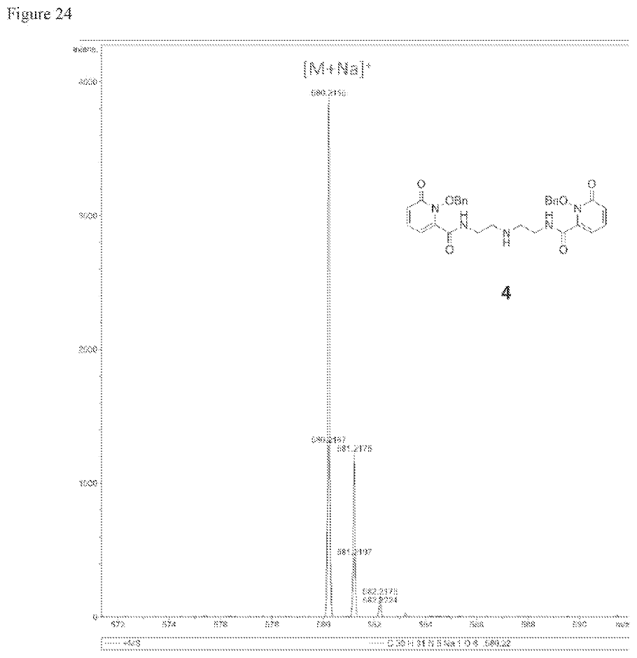

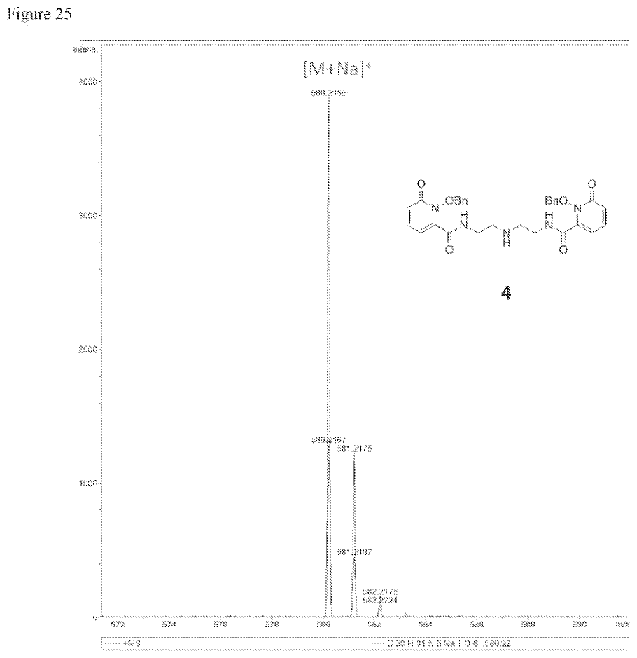

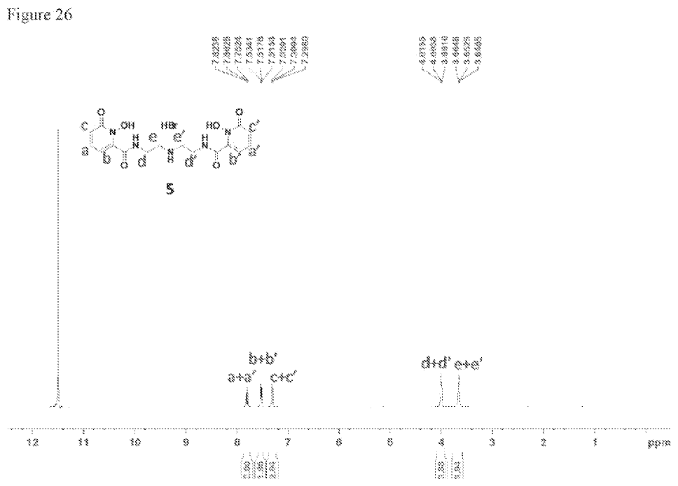

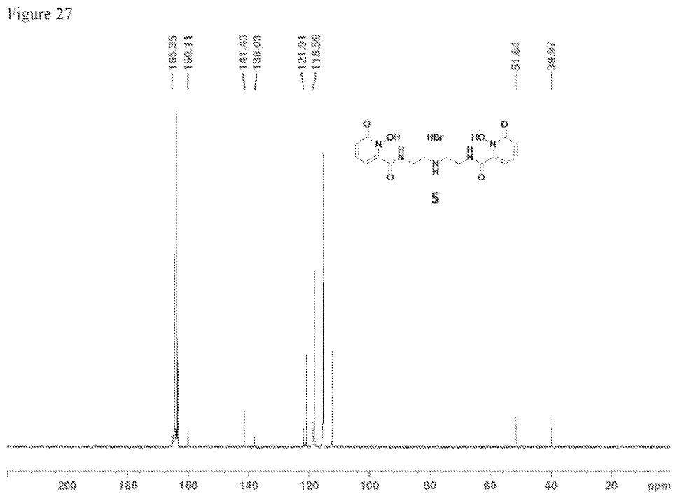

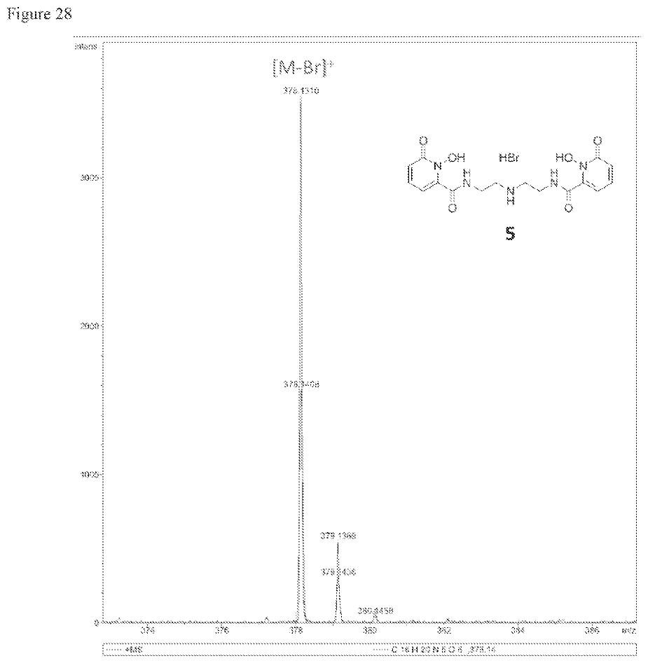

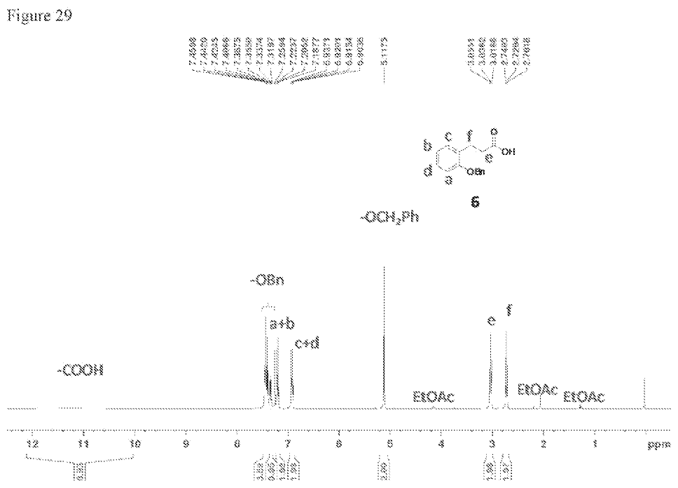

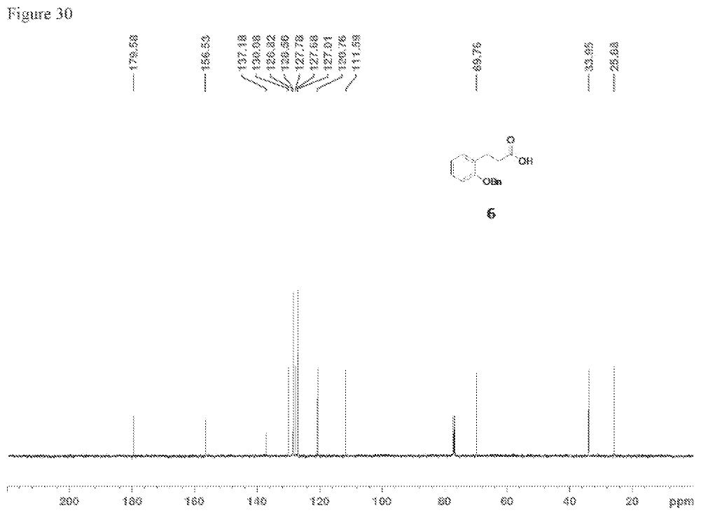

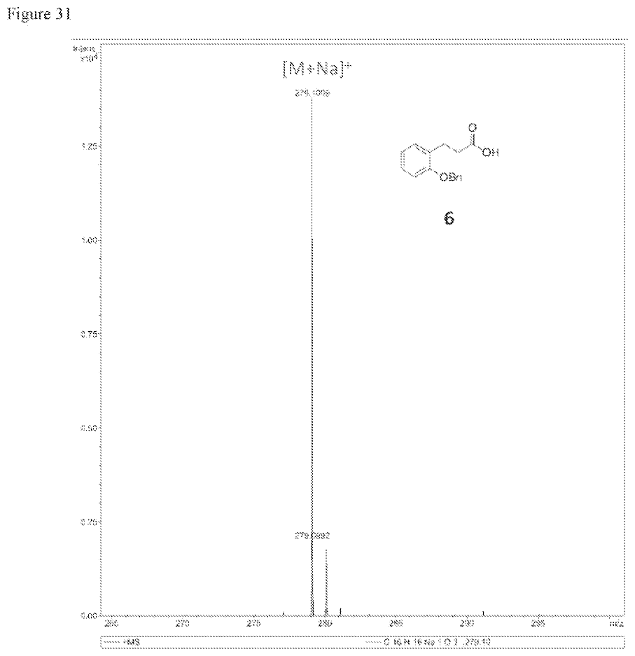

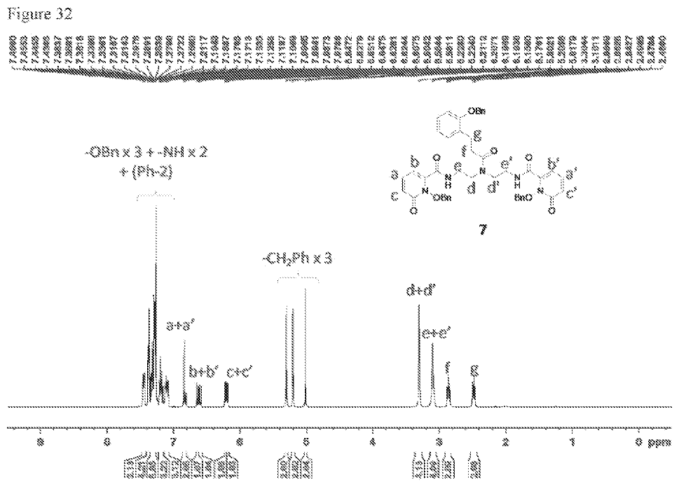

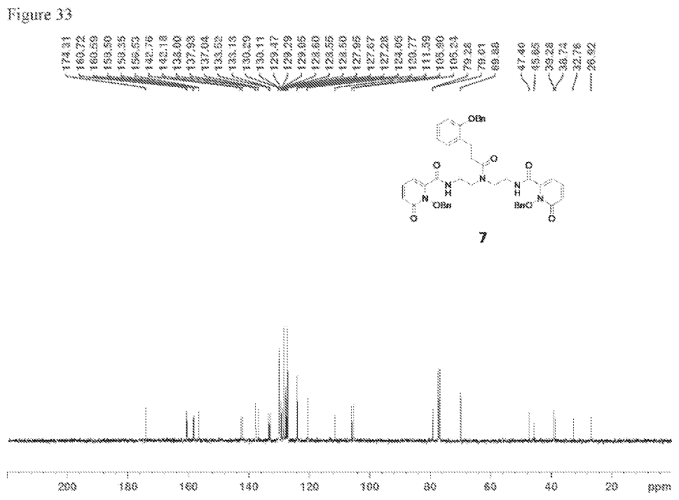

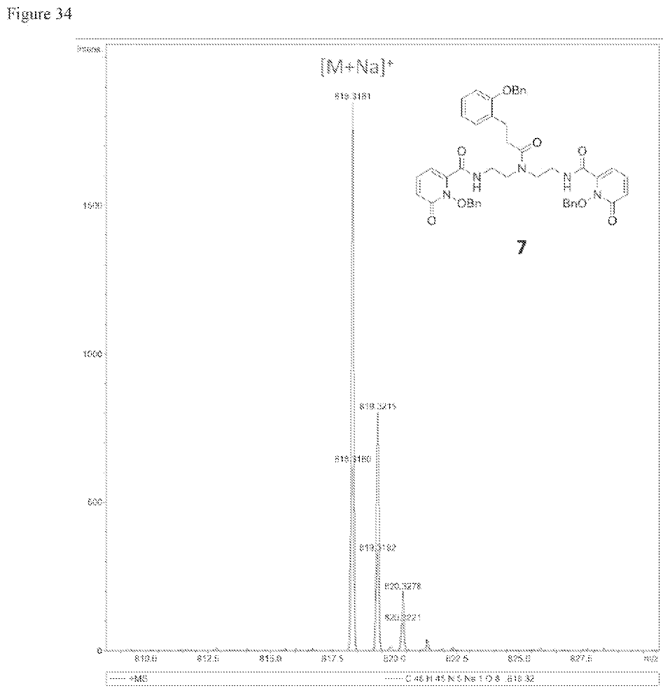

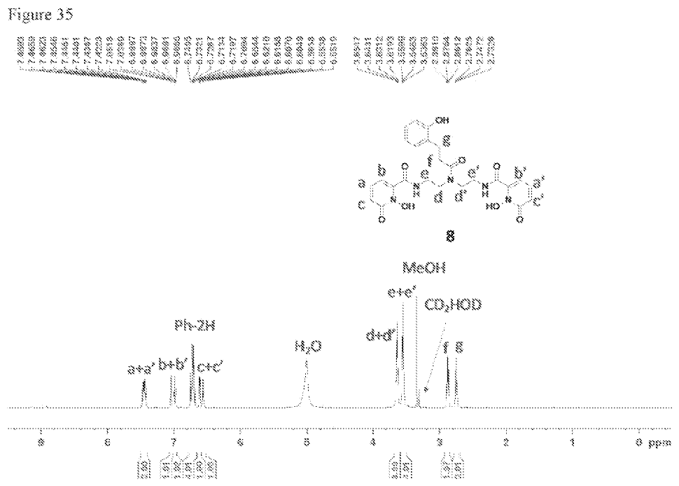

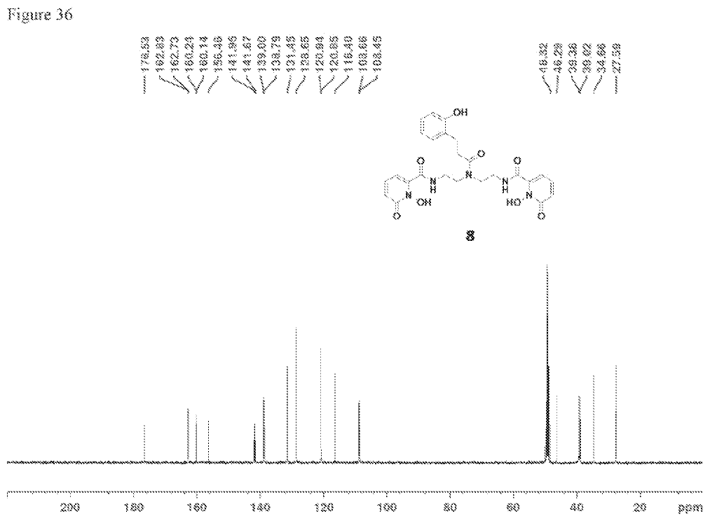

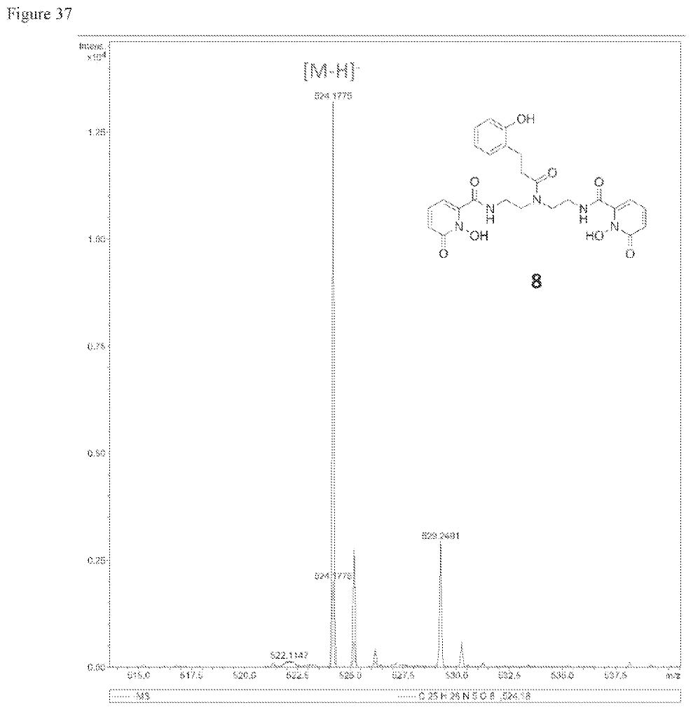

The indicator displacement assays (IDA) was evaluated by both UV-visible and fluorescence spectroscopy. A 20-fold turn-on fluorescence was observed upon gradual addition of 1 equivalent of orthophosphate (FIG. 3A). The fluorescence titrations (FIG. 3 and FIG. 19) of both receptors were best fitted to a 1:1 binding model from which the equilibrium constants were derived (Table 1). This 1:1 stoichiometry was determined first by evaluating the fit of the titrations and subsequently confirmed by Job's plots (FIGS. 15 and 16 for Fe^{III}—HOPO-fluo and Fe^{III}—HOPO-PhO-fluo, respectively). Interestingly, the use of a tetradentate ligand in Fe^{III}—HOPO-fluo does not appear to favor coordination of two phosphate anions to the metal center. The two receptors display similar turn-on response (20-fold at 1 equivalent) and similar equilibrium constants for phosphate: 8.8×10⁵ M⁻¹ and 1.1×10⁶ M⁻¹ for Fe^{III}—HOPO-fluo and Fe^{III}—HOPO-PhO-fluo respectively. This similarity in both turn-on response and apparent equilibrium constants could be attributed to the comparable core structure of both receptors. Interestingly, the extra phenolate podand of 2 does not appear to affect displacement of the fluorescein moiety by phosphate. A likely coordinated solvent molecule appears to have similar effect.

The limit of detection (LOD) of phosphate by the two Fe^{III} receptors, commonly estimated as three times the standard deviation of measurement (3σ), are 3.5 μM and 4.1 μM for Fe^{III}—HOPO-fluo (1) and Fe^{III}—HOPO-PhO-fluo (2), respectively (Table S1). Although not quite as sensitive as prior Eu^{III} probes,^{21,22} these iron receptors are sensitive enough to detect problematic phosphate levels in eutrophic samples (2-10 μM).^{49,50}

TABLE 1
Apparent equilibrium constants of Fe^{III}—HOPO-fluo (1) and
Fe^{III}—HOPO—PhO-fluo (2) with orthophosphate.

| | Kₐ (M⁻¹) |
|---|---|
| Fe^{III}—HOPO-fluo | 8.8 ± 3.4 × 10⁵ |
| Fe^{III}—HOPO—PhO-fluo | 1.1 ± 0.5 × 10⁶ |

The selectivity of the two iron receptors for phosphate over competing anions commonly found in environmental samples was also evaluated by fluorescence spectroscopy. As shown in the white bars of FIG. 4, the fluorescence intensity of both probes is not affected by the addition of 1 equivalent of common competing anions including halides, sulfate, and nitrate. Subsequent addition of 1 equivalent of phosphate restores the luminescence of the indicator (FIG. 4, grey bars) further indicating that these competing anions do not interfere with detection of phosphate. Interestingly, Fe^{III}—HOPO-fluo is more selective over bicarbonate and acetate than Fe^{III}—HOPO-PhO-fluo. A more sterically hindered recognition site therefore does not appear to generate higher selectivity for the targeted anion.

Uniquely, and importantly, both Fe^{III}—HOPO-fluo (1) and Fe^{III}—HOPO-PhO-fluo (2) are selective for phosphate over pyrophosphate. Whereas numerous probes selective for pyrophosphate over phosphates have been described in the literature,³⁹⁻⁴¹ it is believed that complexes 1 and 2 are unique in their reverse selectivity for phosphate over pyrophosphate. This selectivity likely stems from the preferred bidentate binding mode of pyrophosphate and likely steric hindrance at the coordination site.^{51,52} Since only one displaceable fluorescein is present, bidentate binding is disfavored. The slightly softer anion-arsenate, also does not displace fluorescein despite its structurally similarity to phosphate. This is an unusual selectivity given as most metal probe for phosphate also respond to arsenate.⁷ As such, these fluorescent iron(III) probes offer a distinctive ability to rapidly monitor the level of the most important phosphorus species causing nutrient pollution in surface water:phosphate.

### SUMMARY

Herein is described non-heme iron(III) complexes, including Fe^{III}—HOPO-fluo and Fe^{III}—HOPO-PhO-fluo, for selective recognition of inorganic phosphate. This is demonstrated via indicator displacement assay. The open coordination sites were sufficiently protected by weakly coordinating fluorescein to prevent dimerization in aerated solutions. Coordination of inorganic phosphate concomitant with displacement of the fluorescein moiety increases the emission of the latter by 20-fold. Uniquely, these probes distinguish themselves from other receptors that function by direct metal coordination in that they are highly selective for phosphate over pyrophosphate. They are also highly selective over common competing endogenous anions such as carbonate, nitrate, sulfate, halides and, unusually, arsenate. The limit of detection of the iron(III) receptors, 3.5 and 4.1 μM for Fe^{III}—HOPO-fluo and Fe^{III}—HOPO-PhO-fluo, respectively, enables detection of phosphate typical of eutrophic water samples. On this basis, the two iron(III) probes enable rapid and facile detection of phosphate in eutrophic samples. It is believed these are the first examples employing non-heme Fe^{III}-based molecular receptors for anions. These results thus provide for the use of inorganic phosphate probes that include iron, an earth abundant and economical element.

### DOCUMENTS

(1) National Oceanic and Atmospheric Administration. What is eutrophication?https://oceanservice.noaa.gov/facts/eutrophication.html (accessed Nov. 9, 2020).
(2) Bricker, S. B. Effects of Nutrient Enrichment in the Nation's Estuaries: A Decade of Change: National Estuarine Eutrophication Assessment Update; NOAA Coastal Ocean Program decision analysis series; no. 26; 328; Silver Spring, MD, 2007.
(3) 5.6 Phosphorus|Monitoring & Assessment|US EPA https://archive.epa.gov/water/archive/web/html/vms56.html (accessed Nov. 23, 2020).
(4) Boyd, C. E. Water Quality: An Introduction, 3rd ed.; Springer International Publishing, 2020.
(5) Rice, E. W.; Baird, R. B.; Eaton, A. D. Standard Methods for the Examination of Water and Wastewater, 23rd ed.; American Public Health Association, American Water Works Association, Water Environment Federation, 2017.
(6) Sundareshwar, P. V.; Morris, J. T.; Pellechia, P. J.; Cohen, H. J.; Porter, D. E.; Jones, B. C. Occurrence and Ecological Implications of Pyrophosphate in Estuaries. Limnol. Oceanogr 2001, 46 (6), 1570-1577.
(7) Raju, M. V. R.; Harris, S. M.; Pierre, V. C. Design and Applications of Metal-Based Molecular Receptors and Probes for Inorganic Phosphate. Chem. Soc. Rev. 2020, 49 (4), 1090-1108.
(8) Meng, Q.; Wu, M.; Shang, Z.; Zhang, Z.; Zhang, R. Responsive Gadolinium(III) Complex-Based Small Molecule Magnetic Resonance Imaging Probes: Design, Mechanism and Application. Coord. Chem. Rev. 2022, 457, 214398.
(9) Macreadie, L. K.; Gilchrist, A. M.; McNaughton, D. A.; Ryder, W. G.; Fares, M.; Gale, P. A. Progress in Anion Receptor Chemistry. Chem 2022, 8 (1), 46-118.
(10) Chen, L.; Berry, S. N.; Wu, X.; Howe, E. N. W.; Gale, P. A. Advances in Anion Receptor Chemistry. Chem 2020, 6 (1), 61-141.
(11) E. Bodman, S.; J. Butler, S. Advances in Anion Binding and Sensing Using Luminescent Lanthanide Complexes. Chem. Sci. 2021, 12 (8), 2716-2734.
(12) Pierre, V. C.; Wilharm, R. K. Design Principles and Applications of Selective Lanthanide-Based Receptors for Inorganic Phosphate. Front. Chem. 2022, 10, 821020.
(13) Sen, B.; Kumar Patra, S.; Rabha, M.; Kumar Sheet, S.; Aguan, K.; Samanta, D.; Khatua,
S. Luminescence Detection of Ag+ and Phosphate Ions by a Ruthenium(II) Complex-Based Multianalyte Probe: A Combined Spectroscopic, Crystallographic, and Theoretical Approach. Eur. J. Inorg. Chem. 2021, 2021 (35), 3549-3560.
(14) Deetz, M. J.; Smith, B. D. Heteroditopic Ruthenium(II) Bipyridyl Receptor with Adjacent Saccharide and Phosphate Binding Sites. Tetrahedron Lett. 1998, 39 (38), 6841-6844.
(15) Kumar, P.; Kumar, S. Detection of Bio-Relevant Metal Ions by Luminescent Ru(II)-Polypyridyl Based Sensors; IntechOpen, 2021.
(16) Marcus, Y. Thermodynamics of Solvation of Ions. Part 5.—Gibbs Free Energy of Hydration at 298.15 K. J. Chem. Soc., Faraday Trans. 1991, 87 (18), 2995-2999.
(17) Beer, P. D.; Gale, P. A. Anion Recognition and Sensing: The State of the Art and Future Perspectives. Angew. Chem. Int. Ed. 2001, 40 (3), 486-516.
(18) Han, M. S.; Kim, D. H. Naked-Eye Detection of Phosphate Ions in Water at Physiological PH: A Remarkably Selective and Easy-To-Assemble Colorimetric Phosphate-Sensing Probe. Angew. Chem. Int. Ed. 2002, 41 (20), 3809-3811.
(19) Tobey, S. L.; Jones, B. D.; Anslyn, E. V. C3v Symmetric Receptors Show High Selectivity and High Affinity for Phosphate. J. Am. Chem. Soc. 2003, 125 (14), 4026-4027.
(20) Harris, S. M.; Nguyen, J. T.; Pailloux, S. L.; Mansergh, J. P.; Dresel, M. J.; Swanholm, T. B.; Gao, T.; Pierre, V. C. Gadolinium Complex for the Catch and Release of Phosphate from Water. Environ. Sci. Technol. 2017, 51 (8), 4549-4558.
(21) Huang, S.-Y.; Qian, M.; Pierre, V. C. A Combination of Factors: Tuning the Affinity of Europium Receptors for Phosphate in Water. Inorg. Chem. 2019, 58 (23), 16087-16099.
(22) Huang, S.-Y.; Qian, M.; Pierre, V. C. The Ligand Cap Affects the Coordination Number but Not Necessarily the Affinity for Anions of Tris-Bidentate Europium Complexes. Inorg. Chem. 2020, 59 (6), 4096-4108.
(23) Ramakrishnam Raju, M. V.; Wilharm, R. K.; Dresel, M. J.; McGreal, M. E.; Mansergh, J. P.; Marting, S. T.; Goodpaster, J. D.; Pierre, V. C. The Stability of the Complex and the Basicity of the Anion Impact the Selectivity and Affinity of Tripodal Gadolinium Complexes for Anions. Inorg. Chem. 2019, 58 (22), 15189-15201.
(24) Schenk, G.; Mitić, N.; Gahan, L. R.; Ollis, D. L.; McGeary, R. P.; Guddat, L. W. Binuclear Metallohydrolases: Complex Mechanistic Strategies for a Simple Chemical Reaction. Acc. Chem. Res. 2012, 45 (9), 1593-1603.
(25) Rodriguez, F.; Lillington, J.; Johnson, S.; Timmel, C. R.; Lea, S. M.; Berks, B. C. Crystal Structure of the Bacillus Subtilis Phosphodiesterase PhoD Reveals an Iron and Calcium-Containing Active Site. J. Biol. Chem. 2014, 289 (45), 30889-30899.
(26) Salvi, F.; Trebacz, M.; Kokot, T.; Hoermann, B.; Rios, P.; Barabas, O.; Köhn, M. Effects of Stably Incorporated Iron on Protein Phosphatase-1 Structure and Activity. FEBS Lett. 2018, 592 (24), 4028-4038.
(27) Kano, K.; Kitagishi, H.; Tamura, S.; Yamada, A. Anion Binding to a Ferric Porphyrin Complexed with Per-O-Methylated β-Cyclodextrin in Aqueous Solution. J. Am. Chem. Soc. 2004, 126 (46), 15202-15210.
(28) Watanabe, K.; Kitagishi, H.; Kano, K. Supramolecular Ferric Porphyrins as Cyanide Receptors in Aqueous Solution. ACS Med. Chem. Lett. 2011, 2 (12), 943-947.
(29) Das, P. K.; Bhattacharya, S. G.; Banerjee, R.; Banerjea, D. Thermodynamic and Kinetic Studies on the Iron(III)-Hydroxamate Interaction in Acid Media. J. Coord. Chem. 1989, 19 (4), 311-320.
(30) Gabric̆ević, M.; Crumbliss, A. L. Kinetics and Mechanism of Iron(III)-Nitrilotriacetate Complex Reactions with Phosphate and Acetohydroxamic Acid. Inorg. Chem. 2003, 42 (13), 4098-4101.
(31) Nishid, Y.; Ito, S. Structures and Reactivities of Several Iron(III) Complexes in the Presence of Hydrogen Peroxide: Relevance to Induction of Tissue Damage Caused by Iron(III) Chelates in Rats. Polyhedron 1995, 14 (17), 2301-2308.
(32) Mizuno, R.; Kawabata, T.; Sutoh, Y.; Nishida, Y.; Okada, S. Oxidative Renal Tubular Injuries Induced by Aminocarboxylate-Type Iron (III) Coordination Compounds as Candidate Renal Carcinogens. Biometals 2006, 19 (6), 675-683.
(33) Gabric̆ević, M.; Crumbliss, A. L. Kinetics and Mechanism of Iron(III)-Nitrilotriacetate Complex Reactions with Phosphate and Acetohydroxamic Acid. Inorg. Chem. 2003, 42 (13), 4098-4101.
(34) Jones, R. D.; Summerville, D. A.; Basolo, Fred. Synthetic Oxygen Carriers Related to Biological Systems. Chem. Rev. 1979, 79 (2), 139-179.
(35) Shikama, K. Stability Properties of Dioxygen-Iron(II) Porphyrins: An Overview from Simple Complexes to Myoglobin. Coord. Chem. Rev. 1988, 83, 73-91.
(36) Huang, X.; Groves, J. T. Oxygen Activation and Radical Transformations in Heme Proteins and Metalloporphyrins. Chem. Rev. 2018, 118 (5), 2491-2553.
(37) Michael A. Kuhn; Brian Hoyland; Scott Carter; Cailan Zhang; Richard P. Haugland. Fluorescent Ion Indicators for Detecting Heavy Metals. In Proc. SPIE 2388; San Jose, CA, 1995; pp 238-244.
(38) Raymond, K. N.; Allred, B. E.; Sia, A. K. Coordination Chemistry of Microbial Iron Transport. Acc. Chem. Res. 2015, 48 (9), 2496-2505.
(39) Guérard, F.; Beyler, M.; Lee, Y.-S.; Tripier, R.; Gestin, J.-F.; Brechbiel, M. W. Investigation of the Complexation of NatZr(IV) and 89Zr(IV) by Hydroxypyridinones for the Development of Chelators for PET Imaging Applications. Dalton Trans. 2017, 46 (14), 4749-4758.
(40) Amsberry, K. L.; Borchardt, R. T. The Lactonization of 2′-Hydroxyhydrocinnamic Acid Amides: A Potential Prodrug for Amines. J. Org. Chem. 1990, 55 (23), 5867-5877.
(41) Arai, Y.; Sparks, D. L. ATR-FTIR Spectroscopic Investigation on Phosphate Adsorption Mechanisms at the Ferrihydrite-Water Interface. J. Colloid Interface Sci. 2001, 241 (2), 317-326.
(42) Rabie, M. S.; Balkees, H. Infrared Study of FeO·OH→Fe2O3 Thermal Transformation. J. Chem. Sci. 1986, 96 (5), 315-320.
(43) Hewkin, D. J.; Griffith, W. P. Infrared Spectra of Binuclear Complexes. J. Chem. Soc. A 1966, No. 0, 472-475.
(44) Aqdim, S.; Ouchetto, M. Elaboration and Structural Investigation of Iron (III) Phosphate Glasses. Adv. Mater. Phys. Chem. 2013, 3 (8), 332-339.
(45) Quin, L. D.; Williams, A. J. Practical Interpretation of P-31 NMR Spectra and Computer Assisted Structure Verification; Advanced Chemistry Development: Toronto, 2004.
(46) Merkx, D. W. H.; Delić, F.; Wierenga, P. A.; Hennebelle, M.; Duynhoven, J. P. M. van. 31P NMR Assessment of the Phosvitin-Iron Complex in Mayonnaise. Magn. Reson. Chem 2019, 57 (9), 540-547.
(47) King, R.; Bonfiglio, R.; Fernandez-Metzler, C.; Miller-Stein, C.; Olah, T. Mechanistic Investigation of Ionization Suppression in Electrospray Ionization. J. Am. Soc. Mass Spectrom. 2000, 11 (11), 942-950.
(48) Mallet, C. R.; Lu, Z.; Mazzeo, J. R. A Study of Ion Suppression Effects in Electrospray Ionization from Mobile Phase Additives and Solid-Phase Extracts. Rapid Commun. Mass Spectrom. 2004, 18 (1), 49-58.
(49) Boyd, C. E. Eutrophication. In Water Quality: An Introduction; Boyd, C. E., Ed.; Springer International Publishing: Cham, 2020; pp 311-322.
(50) Boyd, C. E. Phosphorus. In Water Quality: An Introduction; Boyd, C. E., Ed.; Springer International Publishing: Cham, 2020; pp 291-309.
(51) Ikotun, O. F.; Marino, N.; Kruger, P. E.; Julve, M.; Doyle, R. P. Coordination Complexes Incorporating Pyrophosphate: Structural Overview and Exploration of Their Diverse Magnetic, Catalytic and Biological Properties. Coord. Chem. Rev. 2010, 254 (7), 890-915.
(52) Gupta, A.; Pratt, R.; Mishra, B. Physicochemical Characterization of Ferric Pyrophosphate Citrate. Biometals 2018, 31 (6), 1091-1099
All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

We claim:

1. An iron complex comprising a compound of formula I 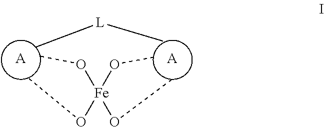
or a salt thereof,
wherein Fe is Fe^{III} or Fe^{III};
each 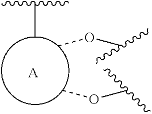
moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b};
X^{a} is phenyl substituted with hydroxy or —O⁻;
W^{a} is a linker^{a} group; and
W^{b} is a linker^{b} group.

2. An iron complex of claim 1 comprising a compound of formula I 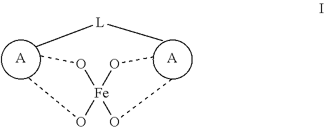
or a salt thereof,
wherein Fe is Fe^{II} or Fe^{III};
each 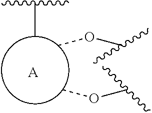
moiety is independently a pyridinone substituted with one hydroxy or —O⁻, wherein the pyridinone is optionally substituted with one or more (C₁-C₆)alkyl, and wherein each dashed bond is independently a single or a double bond;
L is a linker, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y;
X^{a} is phenyl substituted with hydroxy or —O⁻; and
W^{a} is a linker^{a} group;
W^{b} is a linker^{b} group; and
Y is a polymer, hydrogel, membrane, nanoparticle, or material.

3. The iron complex of claim 1, wherein the linker^{a} group and the linker^{b} group are further optionally substituted with Y, wherein Y is a polymer, hydrogel, membrane, nanoparticle, or material.

4. The iron complex of claim 1, wherein each 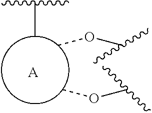
moiety is independently selected from the group consisting of: 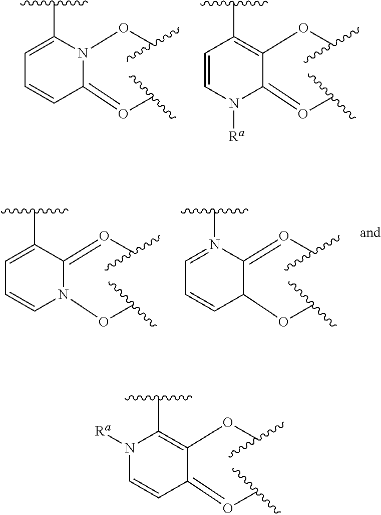
and
wherein each R^{a} is independently (C₁-C₆)alkyl.

5. The iron complex of claim 1, wherein each 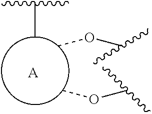
moiety is

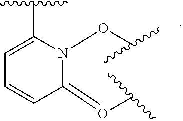

6. The iron complex of claim 1, wherein each 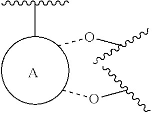
moiety is

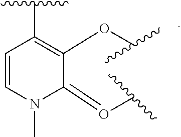

7. The iron complex of claim 1, wherein each 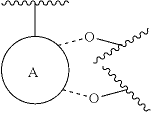
moiety is

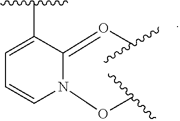

8. The iron complex of claim 1, wherein each 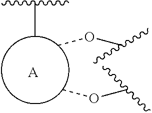
moiety is

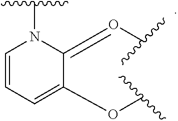

9. The iron complex of claim 1, wherein each 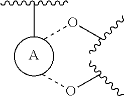
moiety is

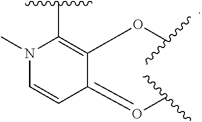

10. The iron complex of any one of claims 1-9, wherein L is a linker that comprises 5-50 non-hydrogen atoms, wherein the non-hydrogen atoms are selected from halo, C, N, S, and O, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

11. The iron complex of any one of claims 1-9, wherein L is a linker that comprises 5-15 non-hydrogen atoms, wherein the non-hydrogen atoms are selected from halo, C, N, S, and O, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

12. The iron complex of any one of claims 1-9, wherein L is a linker that comprises a branched or unbranched, saturated or unsaturated, hydrocarbon chain, having from 3 to 15 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from (C₁-C₄)alkyl, (C₁-C₆)alkoxy, oxo (═O), and halo, wherein each R^{a} is independently H or (C₁-C₆)alkyl, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b}.

13. The iron complex of any one of claims 1-9, wherein L is 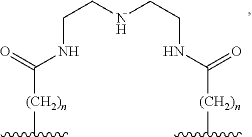
wherein the L is optionally substituted with —W^{a} or —C(═O)(C₁-C₆)alkyl-X^{a}, and wherein the —C(═O)(C₁-C₆)alkyl-X^{a} is optionally substituted with —W^{b} and wherein n is 0 or 1.

14. The iron complex of claim 1 comprising a compound of formula Ia′ 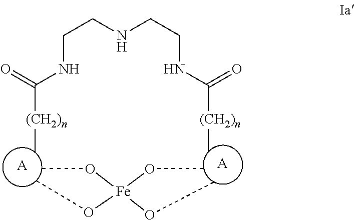
or a salt thereof,
wherein n is 0 or 1, R is H, —W^{a}, or —C(═O)(C₁-C₆)alkyl-X^{a}, and wherein the —C(═O)(C₁-C₆)alkyl-X^{a} is optionally substituted with —W^{b}.

15. The iron complex of claim 14, wherein R is H.

16. The iron complex of claim 14, wherein R is —W^{a}.

17. The iron complex of claim 14, wherein R is —C(═O)(C₁-C₆)alkyl-X^{a}, wherein the —C(═O)(C₁-C₆)alkyl-X^{a} is optionally substituted with —W^{b}.

18. The iron complex of claim 1 comprising a compound of formula Ib′ 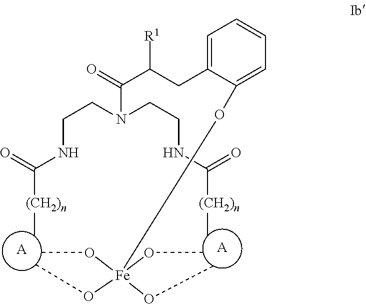
or a salt thereof,
wherein n is 0 or 1; R¹ is H or —W^{b}.

19. The iron complex of any one of claims 1-14 or 16-18, wherein each W^{a} and W^{b} independently comprises 2-50 non-hydrogen atoms, wherein the non-hydrogen atoms are selected from halo, C, N, S, and O.

20. The iron complex of any one of claims 1-14 or 16-18, wherein each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl.

21. The iron complex of any one of claims 1-14 or 16-18, wherein each W^{a} and W^{b} independently comprises a branched or unbranched, saturated or unsaturated, hydrocarbon chain, having from 1 to 20 carbon atoms wherein one or more of the carbon atoms is optionally replaced independently by —O—, —S, or —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl, wherein the chain is substituted with one or more reactive groups.

22. The iron complex of any one of claims 1-14 or 16-18, wherein each W^{a} and W^{b} independently comprises a branched or unbranched, saturated or unsaturated, hydrocarbon chain, having from 1 to 10 carbon atoms wherein one or more of the carbon atoms is optionally replaced independently by —O—, —S, or —N(R^{a})—, and wherein the chain is optionally substituted with one or more (e.g. 1, 2, 3, 4, 5 or more) substituents independently selected from oxo (C═O), (C₁-C₄)alkyl, (C₁-C₆)alkoxy, hydroxy, and halo, wherein each R^{a} is independently H or (C₁-C₆)alkyl, wherein the chain is substituted with one or more reactive groups.

23. The iron complex of claim 21 or 22, wherein each reactive group is independently an amine, thiol, hydroxy, amide or ester.

24. The iron complex of any one of claims 1-14 or 16-18, wherein
W^{a} is 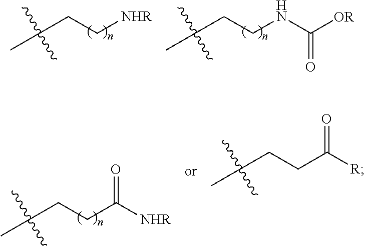
W^{b} is 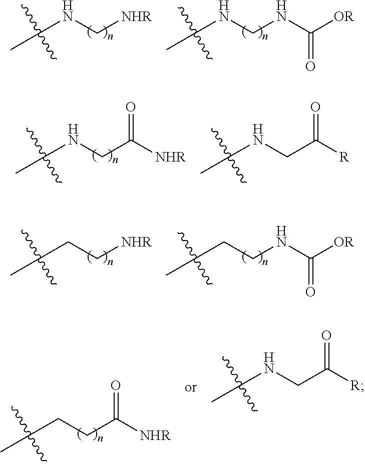
and
R is H or (C₁-C₆)alkyl; and
n is 1, 2, 3, 4, 5, 6, 7, 8, 9, or 10.

25. The iron complex of claim 1, wherein the compound of formula I is: 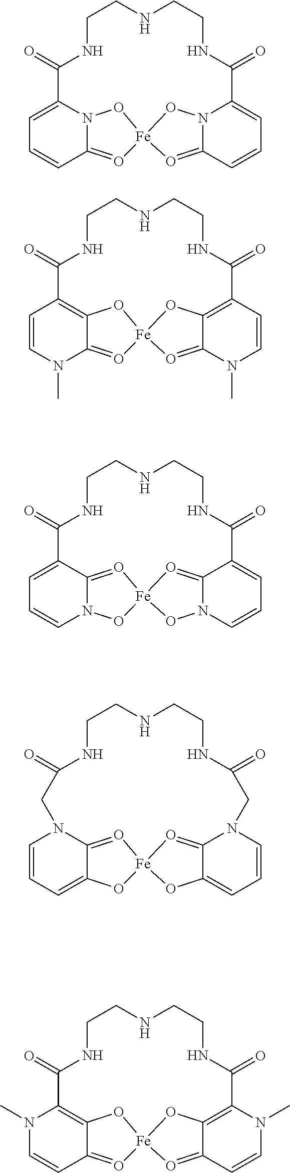 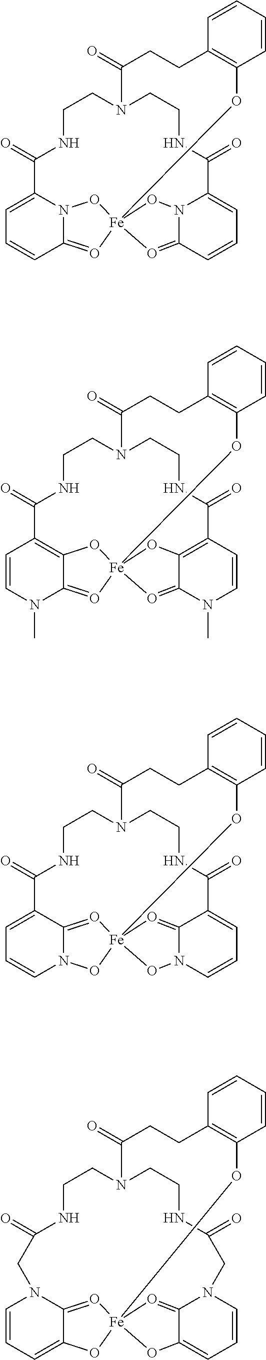 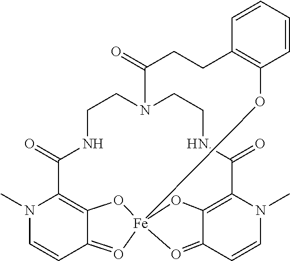
or a salt thereof.

26. A material or device comprising one or more an iron complexes or salts thereof as described in any one of claims 1-25,

27. The material or device of claim 26, wherein the material or device is attached to linker W^{a} or W^{b}.

28. The material or device of claim 26 or 27, comprising one or more iron complexes selected from 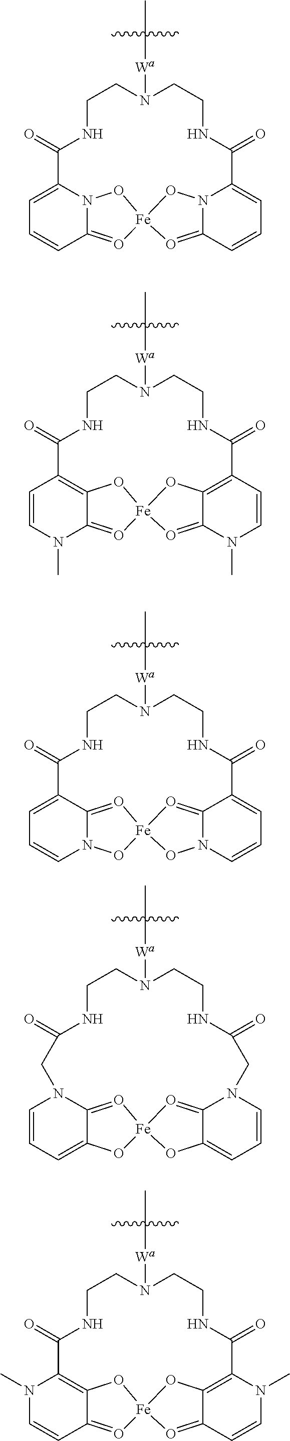 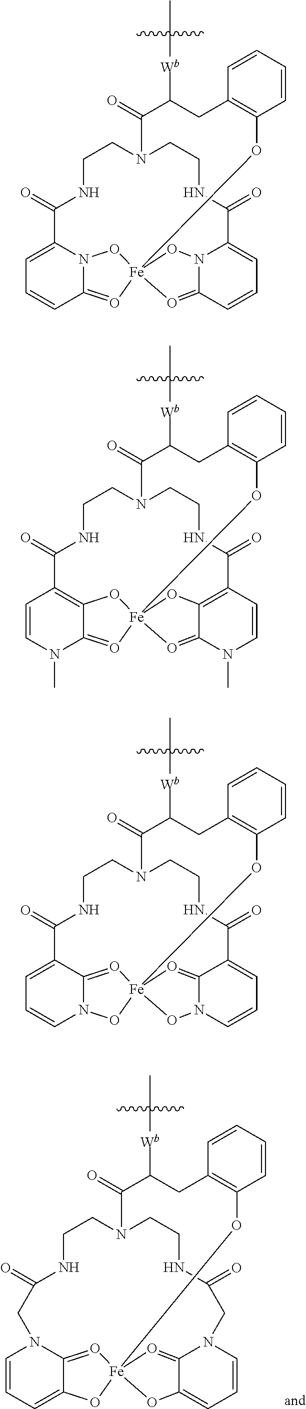 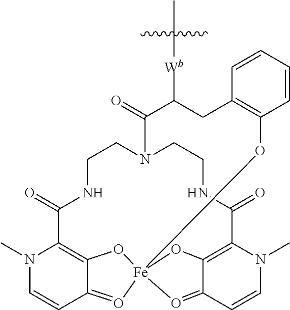
or a salt thereof.

29. The iron complex or salt thereof of any one of claims 26-28, wherein
W^{a} is 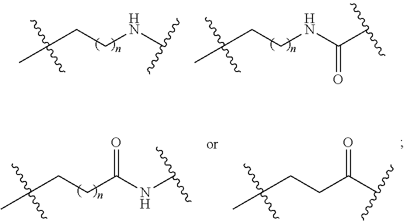
W^{b} is 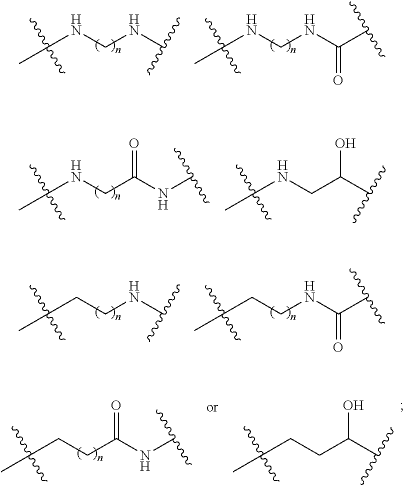
and
n is 1, 2, 3, 4, 5, 6, 7, 8, 9, or 10.

30. The iron complex or salt thereof of claim 2, wherein the compound of formula I is 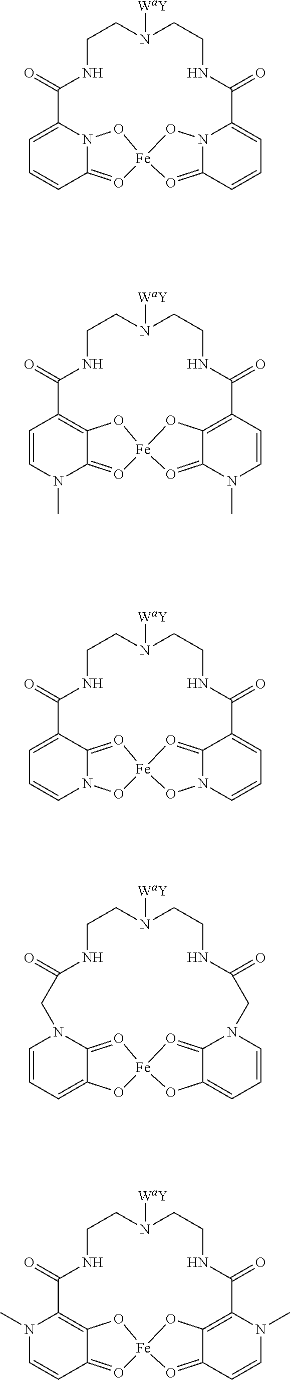 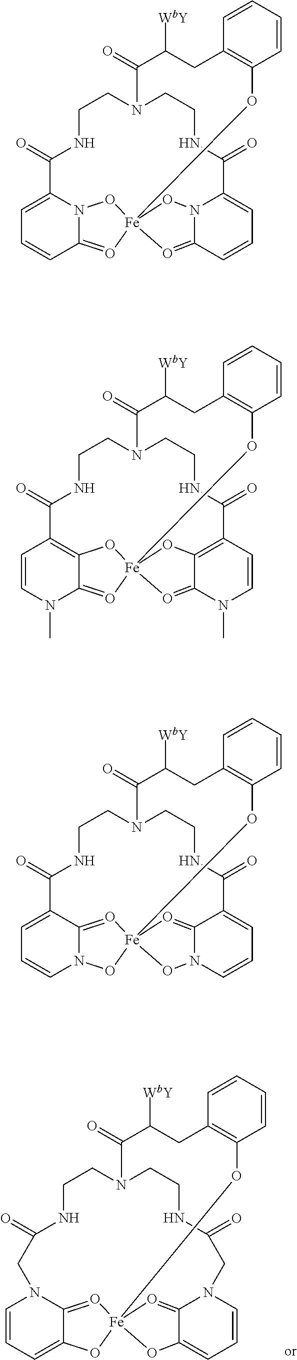 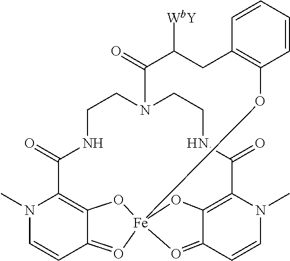
or a salt thereof.

31. The iron complex of claim 1, wherein the compound of formula I is: 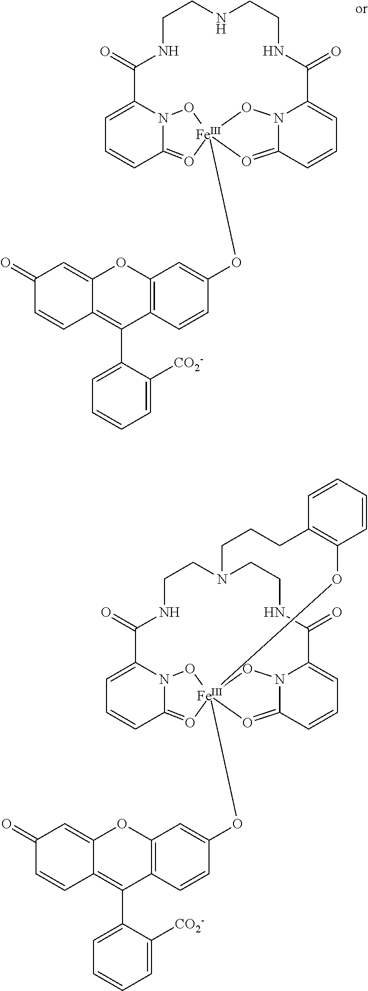
or a salt thereof.

32. The iron complex of any one of claims 1-31 further comprising a weak binding ligand.

33. The iron complex of claim 32, wherein the weak binding ligand is fluorescein.

34. A method to detect inorganic phosphate comprising contacting the phosphate with an iron complex as described in any one of claims 1-33.

35. The method of claim 34 wherein the phosphate is selectively detected in the presence of other anions.

36. The method of claim 35, wherein the other anions are selected from the group consisting of carbonate, nitrate, sulfate, halides, arsenate and pyrophosphate.

37. The method of any one of claims 34-36, wherein the phosphate is contacted with the iron complex as a liquid sample at about neutral pH.

38. The method of claim 37, wherein the liquid sample is sample obtained from a body of water.

39. The method of claim 37 or 38, where the liquid sample is a eutrophic sample.

40. The method of any one of claims 34-39, wherein the phosphate is detected by fluorescence sensing by an indicator displacement assay.

41. A method to remove inorganic phosphate from an aqueous mixture or solution comprising contacting the aqueous mixture or solution with an iron complex as described in any one of claims 1-33.

42. The method of claim 41, wherein the aqueous mixture or solution is waste water.

43. A method to treat hyperphosphatemia in a mammal in need thereof comprising contacting the blood of the mammal in need thereof, with an iron complex as described in any one of claims 1-33.

44. The method of claim 43, wherein the mammal has chronic kidney disease.

45. A ligand of formula II 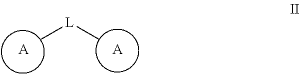
wherein:
each 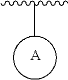
moiety is independently a pyridinone substituted with one hydroxy or —O⁻, wherein the pyridinone is optionally substituted with one or more (C₁-C₆);
L is a linker, wherein the linker is optionally substituted with —W^{a}—Y or —C(═O)(C₁-C₆)alkyl-X^{a}, and wherein the —C(═O)(C₁-C₆)alkyl-X^{a} is optionally substituted with —W^{b}—Y;
X^{a} is phenyl substituted with hydroxy or —O⁻;
W^{a} is a linker^{a} group;
W^{b} is a linker^{b} group; and
Y is a polymer, hydrogel, membrane, nanoparticle, or material.

46. A ligand of formula II 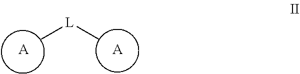
each 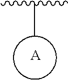
moiety is independently a pyridinone substituted with one hydroxy or —O⁻, wherein the pyridinone is optionally substituted with one or more (C₁-C₆)alkyl;
L is a linker, wherein the linker is optionally substituted with —W^{a} or —C(═O)(C₁-C₆)alkyl-X^{a}, and wherein the —C(═O)(C₁-C₆)alkyl-X^{a} is optionally substituted with —W^{b},
X^{a} is phenyl substituted with hydroxy or —O⁻;
W^{a} is a linker^{a} group; and
W^{b} is a linker^{b} group.

47. The ligand of claim 45 or 46, wherein A, L, X^{a}, W^{a}, W^{b} and Y are as defined in any one of claims 1-33.

48. The compound or salt thereof of formula I as defined in any one of claims 1-33, wherein the iron (Fe (Fe^{II} or Fe^{III} is absent)) is absent.
